(19) United States
(12) Patent Application Publication
(10) Pub. No.: US 2026/0176374 A1
(43) Pub. Date: Jun. 25, 2026

(54) SYNTHETIC ANTIBODY AGONISTS OF ERYTHROPOIETIN RECEPTOR

(71) Applicant: EPOK THERAPEUTICS INC., Toronto (CA)

(72) Inventors: Sachdev SIDHU, Toronto (CA); Ning YANG, Toronto (CA); Jarrett ADAMS, Toronto (CA); Rodilia CARDARELLI, Toronto (CA)

(21) Appl. No.: 19/129,774

(22) PCT Filed: Nov. 14, 2023
(86) PCT No.: PCT/IB23/61477

§ 371 Date: May 14, 2025

Related U.S. Application Data
(60) Provisional application No. 63/383,584, filed on Nov. 14, 2022.

(30) Foreign Application Priority Data
Nov. 15, 2022 (IT) 102022000023499

(51) Int. Cl. C07K 16/32; A61K 38/00; C07K 14/005

(52) U.S. Cl. CPC C07K 16/32; A61K 38/00; C07K 14/005; C07K 2317/34; C07K 2317/55; C07K 2317/565; C07K 2317/92

(57) ABSTRACT

It forms an object of the present invention erythropoietin receptor (EPOR) binding sites and EPOR agonists and antagonists in the diagnosis and treatment of disease, particularly kidney disease and anaemias arising from cancer treatment, or inherited syndromes. More particularly, the present invention is directed to synthetic EPOR antibody constructs, particularly diabodies.

The present invention relates to erythropoietin receptor (EPOR) binding sites and EPOR agonists and antagonists in the diagnosis and treatment of disease, particularly kidney disease and anaemias arising from cancer treatment, or inherited syndromes. More particularly, the present invention is directed to synthetic EPOR antibody constructs, particularly diabodies. These EPOR agonists represent a novel therapeutic option for patients with chronic kidney disease, treatment-related anaemia, and inherited EPO deficiency.

### BACKGROUND

Human erythropoietin (EPO) is a growth factor that promotes red blood cell generation. It binds to the human EPO receptor (hEPOR) on the red blood cell progenitors and activates an intracellular signalling cascade involved in erythropoiesis. Naturally, it is produced by the peritubular cells of the kidney in response to hypoxia. Patients with chronic kidney disease (CKD) very often have reduced EPO production and consequently suffer from anaemia, a condition of red blood cell deficiency. As a result, patients require frequent administration of recombinant EPO or other erythropoiesis-stimulating agents (ESAs) to maintain normal red blood cell production. The most common ESAs are epoetin alfa and darbepoetin alfa, which are given to patients at a dosing interval of 2 to 3 times weekly and every 1 to 2 weeks, respectively.

Erythropoiesis begins with lineage commitment of hematopoietic stem and progenitor cells (HSPCs) into the erythroid lineage. As HSPCs differentiate into erythroid progenitors, they begin to express EPOR and become responsive to circulating EPO that promotes further proliferation and eventual maturation into differentiated red blood cells. The binding of hEPO to hEPOR occurs via high- and low-affinity binding sites (Sites 1 and 2, respectively), inducing conformational changes in the receptor, followed by the activation of downstream signalling cascades. Inactive hEPOR is thought to be expressed at the cell surface as a pre-formed dimer in the absence of ligand. hEPO binding proceeds by an asymmetrical association, first via the high affinity hEPO site 1 followed by the lower affinity site 2 interaction, which stimulates a reorientation of hEPOR monomers within the dimeric complex and subsequent activation of the JAK2 receptor-associated kinase.

In addition to CKD, anaemia is a common complication that affects ˜40% of cancer patients and ˜90% of patients receiving chemotherapy. While recombinant hEPO is an effective treatment for cancer-associated anaemia, its adverse effects on the disease recurrence and patient survival have raised concerns and precluded its use in many oncology patients. hEPO has deleterious effects on cancer patients, at least in part, through non-erythropoietic signalling pathways such as Ephrin B4 receptor (EPHB4) and IL-3R common β-subunit (CD131). hEPO may also increase cancer growth through increased oxygen delivery to hypoxic areas within the tumour. The pleiotropic effects of EPO highlight the need and the opportunity not only for the development of EPOR-stimulating, erythropoiesis-specific therapies but also ways to selectively inhibit EPO signalling mechanisms that promote cancer.

There remains to be identified agonists and antagonists of EPOR that meet the challenges noted above that aid in the diagnosis and treatment of diseases, particularly anaemias of cancer and other disease conditions.

### SUMMARY OF THE INVENTION

The present invention provides a novel epitope on the hEPOR. Said epitope provides for selective activation of the erythropoiesis-specific effects of the hEPOR without adverse effects, such as, but not limited to, activating other cell surface receptors such as EPHB4 and CD131 that may have oncogenic roles.

The present invention also provides antibodies and polypeptide sequences capable of selectively binding with the same high affinity to human EPOR with little affinity for murine EPOR, (e.g., see FIGS. 1A, 2, and 9). Said antibodies and polypeptide sequences are capable of acting as hEPO agonists and antagonists.

An embodiment of the present invention is the use of hEPOR agonists as diagnostic agents and therapeutic agents for the treatment of disease, including CKD, cancer-associated or treatment-related anaemias and inherited anaemias.

An aspect of the invention is directed to highly selective hEPOR agonists, including, in a preferred embodiment, synthetic binding molecules, such as, for example, antibodies, diabodies, higher order molecules such as tetravalents. In a more preferred embodiment of the present invention there is provided synthetic diabodies that act as hEPOR agonists.

An aspect of the invention is directed to highly selective hEPOR antagonists, including, in a preferred embodiment, synthetic binding molecules, such as, for example, antibodies. In a more preferred embodiment of the present invention there is provided synthetic antibodies that act as hEPOR antagonists.

An aspect of the present invention is directed to an hEPOR activation region described herein. According to one embodiment and referring to the NCBI Reference Sequence: NP_000112.1, reported in FIG. 9E (SEQ ID NO: 29), the human hEPOR activation region according to the present invention includes a first region comprising residues 82, 84 to 88, and 90 (numbering refers to SEQ ID NO: 29). According to another embodiment, the human hEPOR activation region according to the present invention includes a second region comprising residues 119, 121, 123, 125, 128, 129, 131, 134, and 136 of hEPOR. According to one embodiment, the human hEPOR activation region according to the present invention includes residues 82, 84 to 88, 90, 119, 121, 123, 125, 128, 129, 131, 134, and 136 of hEPOR.

Another aspect of the present invention is directed to an antibody that binds to a hEPOR activation region described herein. According to one embodiment, the preferred antibody according to this invention binds to residues 82, 84 to 88, and 90 of hEPOR. According to one embodiment, a preferred antibody according to this invention binds to residues 119, 121, 123, 125, 128, 129, 131, 134, and 136 of human EPOR. According to one embodiment, a more preferred antibody according to this invention binds to residues 82, 84 to 88, 90, 119, 121, 123, 125, 128, 129, 131, 134, and 136 of human EPOR. According to another embodiment, the functional epitope of an antibody according to this invention includes residues 82, 84 to 88, 90, 119, 121, 123, 125, 128, 129, 131, 134, and 136 of human EPOR.

According to one embodiment, an antibody of this invention contacts an EPO activation site of the EPOR.

According to one embodiment, an hEPOR activation region of this invention comprises an amino acid sequence of hEPOR comprising QEDEPWL (SEQ ID NO: 1). According to one embodiment, an hEPOR activation region of this invention comprises an amino acid sequence of hEPOR comprising PERTSGPHV (SEQ ID NO. 2). According to a preferred embodiment, an hEPOR activation region of this invention comprises a first amino acid sequence of hEPOR comprising QEDEPWL (SEQ ID NO: 1), a second amino acid sequence of hEPOR comprising PERTSGPHV (SEQ ID NO. 2).

According to another embodiment, the antibody further comprises a CDR-L1 having a contiguous amino acid sequence X1X2X3X4X5, wherein:
X1 is S, D or T;
X2 is V or an aliphatic amino acid;
X3 and X4 are D, E, G, H, K, N, Q, R, or S; and
X5 is A or an aliphatic amino acid.

According to another embodiment, the antibody further comprises a CDR-L2 having a contiguous amino acid sequence X1X2X3X4X5X6X7, wherein:
X1 is S or T;
X2 is A, D or aliphatic amino acid;
X3 and X4 are D, E, G, H, K, N, Q, R, or S;
X5 is L, D or aliphatic amino acid;
X6 is Y or polar amino acid; and
X7 is S, D or T.

According to another embodiment, the antibody further comprises a CDR-L3 having a contiguous amino acid sequence X1X2X3X4X5X6, wherein:
X1 is S, F, T, A, I, or P;
X2 is S, P, T, C, A, F, Y, G, R, or D;
X3 is Y, R, P, S, D, R, H, F, N, I, G, P, E, Q or T;
X4 is S, F, A, G, V, Y, P, T, or N;
X5 is L, P or aliphatic amino acid; and
X6 is I, F or hydrophobic amino acid.

According to another embodiment, the antibody further comprises a CDR-L3 having a contiguous amino acid sequence X1X2X3X4X5, wherein:
X1 is A, D, I, S, T, G, V, or P;
X2 is Y, N, L, D, H, F, S, or V;
X3 is W, S, G, R, L, P, K, or E;
X4 is L, P or aliphatic amino acid; and
X5 is I, F or hydrophobic amino acid.

According to another embodiment, the antibody further comprises a CDR-H1 having a contiguous amino acid sequence X1X2X3X4X5X6, wherein:
X1 is L or aliphatic amino acid;
X2 is Y, S, N, G, D, H, R, F, T, Q, K, P, E, or I;
X3 is S, A, F, Y, R, N, G, T, or H;
X4 is Y, F, H, S, or N;
X5 is Y, A, F, V, L, G, P, T or an aliphatic or aromatic amino acid; and
X6 is I, M or hydrophobic amino acid.

According to another embodiment, the antibody further comprises a CDR-H1 having a contiguous amino acid sequence X1X2X3X4X5X6, wherein:
X1 is L, F or aliphatic amino acid;
X2 is Y, S, N, G, D, H, R, F, T, D, P, or I;
X3 is S, A, F, Y, R, N, G, T, D, or H;
X4 is Y, S or F;
X5 is Y, A, F, V, L, G, P, T, S or aliphatic or aromatic amino acid; and
X6 is I, M or hydrophobic amino acid.

According to another embodiment, the antibody further comprises a CDR-H2 having a contiguous amino acid sequence X1X2X3X4X5X6X7X8X9X10, wherein:
X1 is S, Y or T;
X2 is I or aliphatic amino acid;
X3 is S, Y, A or polar amino acid;
X4 is P or aliphatic amino acid;
X5 is Y, H, F or polar amino acid;
X6 is Y, S, H or polar amino acid;
X7 is S, T, D or G;
X8 is Y, F or polar amino acid;
X9 is T, D or S amino acid; and
X10 is Y, S or polar amino acid.

According to another embodiment, the antibody further comprises a CDR-H3 having a contiguous amino acid sequence X1X2X3X4X5X6, wherein:
X1 is H, R or N;
X2 is G, A, V or S;
X3 is Y, F, or H;
X4 is G, S, I, V, A, T or aliphatic amino acid; and
X5 is A, G or aliphatic amino acid; and
X6 is M, L or hydrophobic amino acid.

According to another embodiment, the antibody further comprises a CDR-H3 having a contiguous amino acid sequence X1X2X3X4X5X6, wherein:
X1 is H, N, or T
X2 is G, A, or S;
X3 is Y, F, or H;
X4 is G, S, A, T or aliphatic amino acid; and
X5 is A or aliphatic amino acid; and
X6 is L or hydrophobic amino acid.

According to another embodiment, the antibody further comprises a CDR-L1 having a contiguous amino acid sequence X1X2X3X4X5, wherein:
X1 is S, or D;
X2 is V;
X3 is S or D
X4 is S; and
X5 is A.

According to another embodiment, the antibody further comprises a CDR-L2 having a contiguous amino acid sequence X1X2X3X4X5X6X7, wherein:
X1 is S;
X2 is A or D;
X3 and X4 are D or S;
X5 is L or D;
X6 is Y; and
X7 is S or D.

According to another embodiment, the antibody further comprises a CDR-L3 having a contiguous amino acid sequence X1X2X3X4X5X6, wherein:
X1 is S;
X2 is S;
X3 is D, H, N, E, Y, or Q;
X4 is S or F;
X5 is L; and
X6 is I or F.

According to another embodiment, the antibody further comprises a CDR-H1 having a contiguous amino acid sequence X1X2X3X4X5X6, wherein:
X1 is L;
X2 is R, D, T, Q, K, S, Y, E or H;
X3 is S;
X4 is Y;
X5 is Y; and
X6 is M.

According to another embodiment, the antibody further comprises a CDR-H2 having a contiguous amino acid sequence X1X2X3X4X5X6X7X8X9X10, wherein:
X1 is S;
X2 is I;
X3 is S, or A;
X4 is P;
X5 is Y, or H;
X6 is Y, or H;
X7 is S, D or G;
X8 is Y;
X9 is T, D or S amino acid; and
X10 is Y.

According to another embodiment, the antibody further comprises a CDR-H3 having a contiguous amino acid sequence X1X2X3X4X5X6, wherein:
X1 is H;
X2 is G;
X3 is Y;
X4 is G or S;
X5 is A; and
X6 is L or M.

According to one embodiment, there is provided an antibody comprising: (a) a CDR-L1 as described herein; (b) a CDR-L2 as described herein; and (c) a CDR-L3 as described herein. According to one embodiment, an antibody of this invention is an antibody comprising: (a) a CDR-H1 as described herein; (b) a CDR-H2 as described herein; and (c) a CDR-H3 as described herein.

According to one embodiment, an aspect of the invention is a synthetic diabody comprising: (a) a CDR-L1 as described herein; (b) a CDR-L2 as described herein; and (c) a CDR-L3 as described herein. According to yet another embodiment of the invention, there is provided a synthetic diabody comprising: (a) a CDR-H1 as described herein; (b) a CDR-H2 as described herein; and (c) a CDR-H3 as described herein.

According to yet another embodiment of the present invention, there is provided a synthetic diabody comprising: (a) a CDR-L1 as described herein; (b) a CDR-L2 as described herein; (c) a CDR-L3 as described herein; (d) a CDR-H1 as described herein; (e) a CDR-H2 as described herein; and (e) a CDR-H3 as described herein.

According to yet another embodiment of the present invention, there is provided a higher order molecule such as tetravalents comprising: (a) a CDR-L1 as described herein; (b) a CDR-L2 as described herein; (c) a CDR-L3 as described herein; (d) a CDR-H1 as described herein; (e) a CDR-H2 as described herein; and (e) a CDR-H3 as described herein.

According to yet another embodiment of the present invention, there is provided the diabodies of the present invention with a linker that promotes the formation of a diabody, preferrable an 18 Å (±2 Å) amino acid linker, more preferably a linker having any amino acid combination of 5 to 15 amino acids, yet more preferably having the sequences GGGGG (SEQ ID NO: 12), IKGGGGGEV (SEQ ID NO: 13), LKVLSRGVV (SEQ ID NO: 14), ISARAGSLV (SEQ ID NO: 15), SKSRAGGEV (SEQ ID NO: 16), LKGGRGGKV (SEQ ID NO: 17), NKGGGGAKV (SEQ ID NO: 18), IKGSSRDDI (SEQ ID NO: 19), MKHAGRGGV (SEQ ID NO: 20), SKGGGGGEV (SEQ ID NO: 21), MKHAGRGGV (SEQ ID NO: 22), LECSDCSGI (SEQ ID NO: 23), yet more preferably having the sequence GGGGG (SEQ ID NO: 12).

According to one embodiment, an antibody of this invention comprises a CDR-L1 comprising the contiguous amino acid sequence SVSSA (SEQ ID NO: 3).

According to one embodiment, an antibody of this invention comprises a CDR-L2 comprising the contiguous amino acid sequence SASSLYS (SEQ ID NO: 4).

According to one embodiment, an antibody of this invention comprises a CDR-L3 comprising the contiguous amino acid sequence SSYSLI (SEQ ID NO: 5). According to one embodiment, an antibody of this invention comprises a CDR-L3 comprising the contiguous amino acid sequence AYWPI (SEQ ID NO: 6). According to one embodiment, an antibody of this invention comprises a CDR-L3 comprising the contiguous amino acid sequence SSYSLF (SEQ ID NO: 24). According to one embodiment, an antibody of this invention comprises a CDR-L3 comprising the contiguous amino acid sequence SSDSLF (SEQ ID NO: 25). According to one embodiment, an antibody of this invention comprises a CDR-L3 comprising the contiguous amino acid sequence SSNFLI (SEQ ID NO: 30), or the contiguous amino acid sequence SSQFLI (SEQ ID NO: 36).

According to one embodiment, an antibody of this invention comprises a CDR-H1 comprising the contiguous amino acid sequence LYSYYI (SEQ ID NO: 7). According to one embodiment, an antibody of this invention comprises a CDR-H1 comprising the contiguous amino acid sequence LSSYYI (SEQ ID NO: 8), or the contiguous amino acid sequence LESYYM (SEQ ID NO: 34), or the contiguous amino acid sequence LRSYYM (SEQ ID NO: 38), or the contiguous amino acid sequence LESYYI (SEQ ID NO: 37), or the contiguous amino acid sequence LDSYYI (SEQ ID NO: 35).

According to one embodiment, an antibody of this invention comprises a CDR-H2 comprising the contiguous amino acid sequence SISPYYSYTY (SEQ ID NO: 9). According to one embodiment, an antibody of this invention comprises a CDR-H2 comprising the contiguous amino acid sequence SISPHYGYTY (SEQ ID NO: 26), or the contiguous amino acid sequence SIAPYHGYTY (SEQ ID NO: 31).

According to one embodiment, an antibody of this invention comprises a CDR-H3 comprising the contiguous amino acid sequence HGYGAM (SEQ ID NO: 10). According to one embodiment, an antibody of this invention comprises a CDR-H3 comprising the contiguous amino acid sequence HSYAAL (SEQ ID NO: 11). According to one embodiment, an antibody of this invention comprises a CDR-H3 comprising the contiguous amino acid sequence HGFGAM (SEQ ID NO: 27). According to one embodiment, an antibody of this invention comprises a CDR-H3 comprising the contiguous amino acid sequence HGYSAM (SEQ ID NO: 28), or the contiguous amino acid sequence HGYGAL (SEQ ID NO: 32).

According to yet another embodiment of the present invention, the antibody further comprises the CDR-L1, CDR-L2 and CDR-L3 of any one of the antibodies of FIGS. 6A to 6E, or of FIG. 13, or of FIG. 16.

According to yet another embodiment, the antibody or antigen-binding portion comprises a CDR-L1 comprising SEQ ID NO: 3, a CDR-L2 comprising SEQ ID NO: 4, a CDR-L3 comprising SEQ ID NO: 30, a CDR-H1 comprising SEQ ID NO: 38, a CDR-H2 comprising SEQ ID NO: 31, a CDR-H3 comprising SEQ ID NO: 32, preferably said antibody is Ab 19429.

According to yet another embodiment, the antibody or antigen-binding portion comprises a CDR-L1 comprises SEQ ID NO: 3, the CDR-L2 comprising SEQ ID NO: 4, the CDR-L3 comprising SEQ ID NO: 25, the CDR-H1 comprising SEQ ID NO: 38, the CDR-H2 comprising SEQ ID NO: 26, the CDR-H3 comprising SEQ ID NO: 28, preferably said antibody is Ab 19113.

According to yet another embodiment, the antibody or antigen-binding portion comprises a CDR-L1 comprising SEQ ID NO: 3, a CDR-L2 comprising SEQ ID NO: 4, a CDR-L3 comprising SEQ ID NO: 30, a CDR-H1 comprising SEQ ID NO: 35, a CDR-H2 comprising SEQ ID NO: 31, a CDR-H3 comprising SEQ ID NO: 32, preferably said antibody is Ab19429 H1D2.

According to yet another embodiment, the antibody or antigen-binding portion comprises a CDR-L1 comprising SEQ ID NO: 3, the CDR-L2 comprising SEQ ID NO: 4, the CDR-L3 comprising SEQ ID NO: 36, the CDR-H1 comprising SEQ ID NO: 38, the CDR-H2 comprising SEQ ID NO: 31, the CDR-H3 comprising SEQ ID NO: 32, preferably said antibody is Ab19429 L3Q3.

According to yet another embodiment, the antibody or antigen-binding portion comprises a CDR-L1 comprising SEQ ID NO: 3, the CDR-L2 comprising SEQ ID NO: 4, the CDR-L3 comprising SEQ ID NO: 5, the CDR-H1 comprising SEQ ID NO: 7, the CDR-H2 comprising SEQ ID NO: 9, the CDR-H3 comprising SEQ ID NO: 10, preferably said antibody is Ab 4636.

According to yet another embodiment, the antibody or antigen-binding portion comprises a CDR-L1 comprising SEQ ID NO: 3, the CDR-L2 comprising SEQ ID NO: 4, the CDR-L3 comprising SEQ ID NO: 6, the CDR-H1 comprising SEQ ID NO: 8, the CDR-H2 comprising SEQ ID NO: 9, the CDR-H3 comprising SEQ ID NO: 11. preferably said antibody is Ab 4635.

According to yet another embodiment, the antibody or antigen-binding portion comprises a CDR-L1 comprising SEQ ID NO: 3, the CDR-L2 comprising SEQ ID NO: 4, the CDR-L3 comprising SEQ ID NO: 5, the CDR-H1 comprising SEQ ID NO: 38, the CDR-H2 comprising SEQ ID NO: 9, the CDR-H3 comprising SEQ ID NO: 10, preferably said antibody is Ab 14949.

According to yet another embodiment, the antibody or antigen-binding portion comprises a CDR-L1 comprising SEQ ID NO: 3, the CDR-L2 comprising SEQ ID NO: 4, the CDR-L3 comprising SEQ ID NO: 25, the CDR-H1 comprising SEQ ID NO: 37, the CDR-H2 comprising SEQ ID NO: 26, the CDR-H3 comprises SEQ ID NO: 28, preferably said antibody is Ab 19113 H1E2.

According to yet another embodiment, the CDR-L1 is located at approximately residues 28 to 38, the CDR-L2 is located at approximately residues 56 to 65; and the CDR-L3 is located at approximately residues 107 to 116. According to another embodiment, the CDR-H1 is located at approximately residues 30 to 39, the CDR-H2 is located at approximately residues 55 to 66; and the CDR-H3 is located at approximately residues 107 to 116.

A further embodiment of the invention is directed to an anti-hEPOR antibody that selectively binds to human EPOR and monkey EPOR, but does not bind to an EPOR from other non-human mammal species. Such antibodies with species specific high binding affinities are particularly useful for preclinical research as well as diagnostic and therapeutic applications.

Also contemplated are variants of the synthetic antibodies with improved binding affinities to hEPOR and monkey EPOR compared to EPOR of other non-human species.

Various forms of the antibody, diabodies, other binding molecules and variants thereof are contemplated herein. For example, the antibody mutant may be a full-length antibody (e.g., having a human immunoglobulin constant region) or an antibody fragment (e.g., a Fab or F(ab′)2) or diabody. Furthermore, the compounds of the present invention may be labelled with a detectable label, immobilized on a solid phase and/or conjugated with a heterologous compound (such as a cytotoxic agent).

Diagnostic and therapeutic uses for the compounds of the present invention are contemplated. In one diagnostic application, the invention provides a method for determining the presence of a protein of interest comprising exposing a sample suspected of containing the protein to the compounds of the present invention and determining binding of the compound to the sample. For this use, the invention may include a kit comprising a compound of the present invention and instructions for using it to detect the protein.

The invention also provides a composition comprising the compounds of the present invention and a pharmaceutically acceptable carrier or diluent. This composition for therapeutic use is sterile and may be lyophilized. Also contemplated is the use of a compound of this invention in the manufacture of a medicament for treating an indication described herein.

The invention further provides a method for treating a mammal, comprising administering an effective amount of the embodiments of the present invention to the mammal. The mammal to be treated in the method may be a nonhuman mammal, e.g., a primate suitable for gathering preclinical data or a rodent (e.g., mouse or rat or rabbit). The nonhuman mammal may be healthy (e.g., in toxicology studies) or may be suffering from a disorder to be treated with the composition or compound of interest. In one embodiment, the mammal is suffering from or is at risk of developing a disorder. In one specific embodiment, the disorder is kidney disease, cancer-associated or treatment-related anaemias, and inherited anaemia syndromes. The amount of composition or compound administered will be a therapeutically effective amount to treat the disorder. In dose escalation studies, a variety of doses of the compound or composition may be administered to the mammal. In another embodiment, a therapeutically effective amount of the composition or compound is administered to a human patient to treat a disorder in that patient. In one preferred embodiment, antibodies of this invention useful for treating kidney disease, cancer-associated or treatment-related anaemias, and inherited anaemias are diabodies. Accordingly, the compounds of the present invention can be used in the manufacture of a medicament for treating kidney disease, cancer-associated or treatment-related anaemias, and inherited anaemias.

### BRIEF DESCRIPTION OF THE DRAWINGS

The foregoing and other objects, features and advantages of the present invention should become apparent from the following description when taken in conjunction with the accompanying drawings. The patent or patent application file contains at least one drawing executed in colour. Copies of this patent or patent application publication with colour drawing(s) may be provided by the office upon request and payment of the necessary fee.

FIGS. 1A to 1C show embodiments of the present invention. A. Naïve binding molecule constructs derived from a diabody phage library as described herein, having binding activity against human EPOR and mouse EPOR and showing activity in a TF-1 proliferations assay. The naïve binding molecule constructs were then cloned in a VL-VH diabody Fc format, expressed in mammalian cells, and purified. All diabody-Fc proteins were used to perform ELISAs with immobilized human (black bars) or mouse (white bars) EPOR. The ability of diabody-Fc proteins to induce proliferation of the human erythroid TF-1 cell line was also measured normalized to EPO (silver bars). B. A schematic showing the diabody format that was used for each construct showing the variable heavy and light chains, the linkages, and constant heavy chains. C. Naïve human EPOR binding molecule constructs derived from selections using library F as previously described were expressed and purified as IgGs similar to previous art (Ref 1, 2). These IgGs (solid lines) were tested for their ability to induce TF-1 cell proliferation compared to EPO (dotted line) over a range of concentrations.

FIGS. 2A and 2B shows binding of naïve diabody-Fc proteins to TF-1 cells using flow cytometry. As negative controls, the goat anti-human Alexa488-conjugated secondary antibody alone was used.

FIGS. 3A to 3E show characterization of preferred embodiments of the present invention. A. The CDR amino acid sequences of the two preferred embodiments, 4636 and 4635 according to IMGT nomenclature. B. Titration ELISAs of the two preferred embodiments binding to immobilized human EPOR. The calculated EC50 values from the two curves are 1.5 and 0.56 for 4636 and 4635, respectively. C. ELISAs were also performed to examine specificity of 4636 and 4635 against two receptors—EPHB4 and CD131—previously shown to bind EPO. D. Epitope binding using competitive BioLayer interferometry (“BLI”) was performed by first immobilizing human EPOR that was used to capture the indicated saturating (200 nM) D-Fc in solution phase. After washing of uncaptured D-Fc, the second competing D-Fc was added and its ability to bind EPOR was measured and normalized. E. Competitive phage ELISA was performed by measuring the binding of the indicated phage-D-Fc to immobilized EPOR in the presence of the indicated EPO concentrations.

FIGS. 4A and 4B show functional activity of preferred embodiments of the present invention. A. Embodiments of the present invention (4636 and 4635) stimulated proliferation of a human erythroid UT-7/EPO cell line (Ref. 3). B. Embodiments of the present invention (4636 and 4635) induced phosphorylation of JAK2, STAT3, STAT5, ERK, and AKT that are downstream effectors of EPOR in a dose-dependent fashion (D-Fc was used at 1 nM, 10 nM, and 100 nM and EPO was used at 1 IU/mL, 10 IU/mL, and 100 IU/mL).

FIG. 5A to 5D show 4636 and 4635 heavy and light chain randomization schemes for the affinity maturation phage libraries.

FIG. 6A to 6D show the amino acid CDR sequences of 4636 (FIGS. 6A and 6B), 4635 (FIGS. 6C and 6D) heavy and light chain affinity maturation clones and their phage ELISA binding data.

FIGS. 7A to 7D show the preferred embodiments from 4636 affinity maturation screens. A. The binding of embodiments compared to 4636 parental D-Fc proteins. B. The calculated EC50 values from the curves in FIG. 7A. C. The stimulation of UT7/EPO cell proliferation by the preferred embodiments compared to parental 4636. D. The calculated EC50 values from the curves in FIG. 7C.

FIGS. 8A to 8D show the structural features of the preferred embodiment 14949 in Fab format bound to EPOR. A. The structure of one EPO molecule bound to two asymmetric EPORs via sites 1 and 2 to trigger activation (1 EER). B. Re-orientation of the 14949-Fab/EPOR ternary structure to accommodate for a preferred 18 Å diabody linker connecting VH and VL domains in its most energetically favourable state using Molecule Operating Environment (MOE) software. The CH1 domain is not shown. EPO was superimposed to show how site 2 is blocked but not site 1. C. The 14949-Fab/EPOR ternary structure is compared to the ternary structure formed by EPO as well as a previously identified EPOR peptide antagonist (1 EBA) that induces receptor dimerization without activation (Ref. 4). D. Structures of EPOR in complex with previously discovered diabodies are shown compared with the EPO-EPOR structure (Ref. 5).

FIGS. 9A to 9E show the paratope of 14949 and the corresponding hEPOR epitope. A. A ribbon diagram showing the interacting interfaces with the binding residues annotated in the hEPOR (dark grey) and the heavy (light grey) and light chains (grey) of 14949. B. Open-book view of the paratope showing the interacting residues (dark grey) in both the heavy (left panel) and light (right panel) chains. C. The sequence of all CDR regions of 14949 (underlined) and interacting residues that are shown in B (shaded grey). All amino acid positions are numbered according to IMGT nomenclature. D. An open-book view of the hEPOR epitope showing the interacting residues (grey). E. A sequence alignment comparing hEPOR to that of M. fascicularis and M. musculus. Site 1 and site 2 residues that bind to EPO are denoted with a bolded (*), shaded (*), or both. 14949-binding residues identical to hEPOR are shaded in light grey while those that are not conserved are shaded in dark grey.

FIGS. 10A to 10E compares the binding of molecule 14949, according to the invention, to that of EPO, state of the art ABT-007, state of the art diabody 305, 310, and 330, scFv-Fc-10, -29, and -15. A. The hEPOR residues that interact with hEPO (light grey) is compared to that of the 14949 epitope (mid-grey) in this structure. Both the high-affinity (left panel) and low-affinity (right panel) are shown. Common resides in both sites are shown in dark grey. B. The prior art ABT-007 epitope is compared to the 14949 epitope with specific binding residues shown in light and mid-grey, respectively. Dark grey denotes hEPOR residues that interact with both ABT-007 and 14949 Fab. C. A comparison of the 14949 epitope (mid-grey) to epitopes of the three previously identified diabodies 305 (4Y5V), 310 (4Y5X), and 330 (4Y5Y) (light grey). Residues that interact with 14949 and each of 305, 310, and 330 are shown in dark grey. D. The 14949 epitope is also compared to that of three prior art scFv-Fc agonistic molecules based upon mutagenesis screens. Residues that, when mutated to Alanine, showed a >50% reduction in scFv-Fc binding to hEPOR that also constituted the 14949 epitope are shown in dark grey. Residues that only affected scFv-Fc binding (>50%) but are not part of the 14949 epitope are shown in light light-grey while amino acids specific for 14949 Fab are denoted in mid-grey. E. A chart summarizing all structural comparisons in A to D.

FIGS. 11A to 11C show different modalities of the preferred 14949 embodiment and their functional characterization. A. A schematic of the different modalities including the diabody-Fc format with the VL and VH linked to the CH2 and CH3 domains. Also shown are inter-diabody and intra-diabody formats with the latter having a covalent linker of (GGGGS)₃. B. A titration ELISA experiment showing similar binding of different formats. C. Proliferation of UT7/EPO cells was measured at various doses of the proteins to determine if format affects agonistic activity.

FIGS. 12A to 12D shows the randomization scheme using 14949 as template. A. CDR-L scheme for sub-library AP229. B. CDR-H scheme for sub-library AP229. C. CDR-L scheme for sub-library AP230. D. CDR-H scheme for sub-library AP230.

FIG. 13 lists the sequences of affinity matured clones that bound hEPOR in a phage ELISA and whose binding was blocked by saturating 14949 D-Fc protein. In grey at positions randomized in the affinity maturation libraries AP229 and AP230. Production yields are listed.

FIGS. 14A to 14C show characterization of affinity matured clones that had higher yields than 14949 parental (except 19432 that had a cryptic N-glycosylation site). A. UT7/EPO cell proliferation assay was performed to examine activity of these clones. EPO and 15033 D-Fc were used as positive and negative controls, respectively. B. Non-specificity ELISAs were performed using a panel of representative antigens to examine non-specific binding of high value clones. C. Affinity measurements using BLI comparing the dissociation constant (Ko) of parental 14949 to 19429 and 19113.

FIG. 15 shows the results of a UT7/EPO cell proliferation assay that measures and compares the agonistic activity of affinity matured 19113 and 19429 VL-VH constructs in both diabody-Fc and IgG formats. Activity of ABT-007 in an IgG1 format was also tested in this assay. hEPO and 15033 D-Fc were used as positive and negative controls, respectively.

FIGS. 16A and 16B, B′ show analysis of variants at individual positions that were not previously randomized in the AP229 and AP230 sub-libraries. A. A table showing the individual mutations that were tested. B, B′. A collection of UT7/EPO cell proliferation assays showing activity of the variants shown in A. In each experiment, EPO and 15033 D-Fc were included as positive and negative controls, respectively.

FIGS. 17A and 17B show the fractionation and analysis of various species based on molecular weight after Protein A purification of the 19429-H1 D2/L3Q3 supernatant. A. A size exclusion chromatogram showing the peaks that eluted off the column. There are three main peaks denoted as 1, 2, and 3. B. Material from Peaks 1, 2, and 3, were then used to perform a UT7/EPO cell proliferation assay to measure the agonistic activity of each peak. EPO and 15033 D-Fc were used as controls.

FIGS. 18A and 18B show the fractionation and analysis of various species based on molecular weight after Protein A purification of the 19113-H1E2 supernatant. A. A size exclusion chromatogram showing the peaks that eluted off the column. Trastuzumab was used as a control. There are three main peaks belonging to 19113-H1E2 denoted as 1, 2, and 3. B. Material from Peaks 1, 2, and 3, was then used to perform a UT7/EPO cell proliferation assay to measure the agonistic activity of each peak using EPO and 15033 D-Fc as controls.

### DETAILED DESCRIPTION OF THE INVENTION

In this disclosure, a number of terms and abbreviations are used. The following definitions of such terms and abbreviations are provided.

As used herein, a person skilled in the relevant art can generally understand the term “erythropoietin” or its abbreviation “EPO” refers to the protein erythropoietin or when used in reference to a nucleic acid, the nucleic acid encoding EPO. As used herein, a person skilled in the relevant art will generally understand the term “erythropoietin receptor” or its abbreviation “EPOR” to refer to the EPO receptor or when used in reference to a nucleic acid, the nucleic acid encoding EPOR.

As used herein, a person skilled in the relevant art may generally understand the term “comprising” to generally mean the presence of the stated features, integers, steps, or components as referred to in the claims, but that it does not preclude the presence or addition of one or more other features, integers, steps, components or groups thereof.

As used herein, a person skilled in the relevant art may generally understand the term “treatment” to generally refer to an approach for obtaining beneficial or desired results. Beneficial or desired results can include, but are not limited to, prevention or prophylaxis, alleviation or amelioration of one or more symptoms or conditions, diminishment of the extent of a disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, a person skilled in the relevant art may generally understand the term “therapeutically effective amount” to be an amount sufficient to effect treatment when administered to a subject in need of treatment. In the case of the embodiments of the present invention, a therapeutically effective amount can include, but is not limited to, an amount that eliminates or reduces the effects of the disease, such as for example, the tumor burden, in a subject.

As used herein, a person skilled in the relevant art may generally understand the term “amino acid sequence” to refer to an amino acid sequence of a naturally or non-naturally occurring protein molecule, “amino acid sequence” and like terms, such as “polypeptide” or “protein”, are not meant to limit the amino acid sequence to the complete, native amino acid sequence associated with the recited protein molecule. Amino acid sequences can be referred to as having an amino (N) terminus and a carboxyl I terminus. Individual amino acids in a peptide or polypeptide can be referred to as “residues” and such residues are numbered sequentially beginning from the N-terminus and increasing towards the C-terminus. The amino acids located generally proximal to the N-terminus are generally referred to as the N-terminal amino acids while those located generally proximal to the C-terminus are referred to as the C-terminus amino acids. It will be understood by a person skilled in the relevant art that the reference to amino acid residues as either N terminus or C-terminus amino acid residues may vary depending on the protein.

As used herein, a person skilled in the relevant art may generally understand that the term “aliphatic amino acid” may comprise one of the following amino acids: alanine, glycine, isoleucine, leucine, proline, valine or methionine; the term “polar amino acid” may comprise one of the following amino acids: serine, threonine, cysteine, asparagine, glutamine or tyrosine; and the term “hydrophobic amino acid” may comprise one of the following amino acids: glycine, alanine, valine, leucine, isoleucine, proline, phenylalanine, methionine and tryptophan.

As used herein, a person skilled in the relevant art may generally understand the terms “nucleic acid molecule encoding”, “DNA sequence encoding,” “RNA sequence encoding,” “mRNA sequence encoding,” “an oligonucleotide having a nucleotide sequence encoding a gene” “polynucleotide having a nucleotide sequence encoding a gene,” “DNA encoding”, “RNA encoding” and similar terminology to generally refer to the order or sequence of nucleotides along a single or double strand of nucleic acid comprising the coding region of a gene or, in other words, the nucleic acid sequence that encodes a gene product. The order of these nucleotides determines the order of amino acids along the polypeptide chain. The coding region may be present in a cDNA, genomic DNA, or RNA form. The oligonucleotide or polynucleotide may be single-stranded (e.g. the sense strand) or double-stranded (e.g. antisense and sense strands). Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in expression vectors may contain endogenous enhancers/promoters, splice junctions, intervening sequences, polyadenylation signals, etc. or a combination of both endogenous and exogenous control elements.

A person skilled in the relevant art will understand that nucleic acid molecules are said to have “5′ ends” and “3′ ends” because mononucleotides are linked via a phosphodiester linkage to make oligonucleotides or polynucleotides in a manner such that the 5′ phosphate of one mononucleotide pentose ring is attached to the 3′ oxygen of its neighbour in one direction. Therefore, an end of an oligonucleotides or polynucleotide, referred to as the “5′ end” if it 5′ phosphate is not linked to the 3′ oxygen of a preceding mononucleotide pentose ring and as the “3′ end” if its 3′ oxygen is not linked to the 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular nucleic acid molecule, discrete elements are referred to as being “upstream” or 5′ of the “downstream” or 3′ elements. As a DNA molecule is typically provided in a double helix, the DNA molecule is said to have a “sense” strand and an “antisense” strand. The sense strand and the antisense strand are said to be reverse complementary in that the 3′ end of the sense strand may anneal to the 5′ end of the antisense strand and the 5′ end of the antisense strand may anneal to the 3′ end of the sense strand. The “sense” strand of the DNA molecule is typically copied into a messenger RNA (mRNA) during transcription. The mRNA made during transcription thus has the same sequence as the sense strand through transcription of the antisense strand so that the eventual protein may be based on the sense version of the DNA molecule. The term “antisense strand” is used in reference to a nucleic acid strand that is complementary to the “sense” strand. The designation (−) (i.e., “negative”) is sometimes used in reference to the antisense strand, with the designation (+) (i.e. “positive”) is sometimes used in reference to the sense.

As used herein, the term “homology” refers to a degree of complementarity. There may be partial homology or complete homology (i.e., identity). As applied to polypeptides, the term “substantial homology” as used herein means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Amino acid sequences may differ by conservative amino acid substitutions. A person skilled in the relevant art will understand the term “conservative amino acid substitutions” to refer to the general interchangeability of residues having chemically similar side chains. For example, a group of amino acids having aliphatic side chains may comprise glycine, alanine, valine, leucine, and isoleucine; a group of amino acids having aliphatic-hydroxyl side chains may comprise serine and threonine; a group of amino acids having amide-containing side chains may comprise asparagine and glutamine; a group of amino acids having aromatic side chains may comprise phenylalanine, tyrosine, and tryptophan; a group of amino acids having basic side chains may comprise lysine, arginine, and histidine; and a group of amino acids having sulphur-containing side chains may comprise cysteine and methionine.

The term “fragment” as used herein in reference to single chain amino acid sequences refers to a polypeptide that may have an amino (N) terminus portion and/or carboxy (C) terminus portion deleted as compared to the native protein, but wherein the remaining amino acid sequence of the fragment is identical to the amino acid sequence of the native protein. It will be understood by a person skilled in the relevant art that the term “fragment” may also refer to a portion of a multi-chain protein molecule (e.g., antibody fragment)

The term “naturally-occurring” or “native” as used herein as applied to an object refers to the fact that an object can be found in nature. For example, a polypeptide or polynucleotide sequence that is present in an organism (including viruses) that can be isolated from a source in nature, and which has not been modified is naturally-occurring. A person skilled in the relevant art would understand that the term “synthetic” references a non-naturally occurring compound.

As used herein, the term “target,” refers to a structure, such as, for example, a nucleic acid or protein molecule, to be identified, detected, characterized or amplified. Thus, the “target” is sought to be sorted out from other structures.

The term “isolated” when used in relation to a nucleic acid or peptide, as in “an isolated oligonucleotide”, “isolated polynucleotide” or “isolated polypeptide”, refers to a nucleic acid or amino acid sequence that is identified and separated from at least one contaminant with which it is ordinarily associated in its natural source. Isolated compounds are present in a form or setting that is different from that in which it is found nature. In contrast, non-isolated compounds, such as nucleic acids or amino acid sequences, are found in the state they exist in nature. For example, a given DNA sequence (e.g., a gene) is found on the host cell chromosome in proximity to neighbouring genes; RNA sequences, such as a specific mRNA sequence encoding a specific protein, are found in the cell as a mixture with numerous other mRNAs that encode a multitude of proteins.

As used herein, the term “portion” when in reference to a nucleotide sequence or an amino acid sequence refers to fragments of that sequence.

As used herein, the term “purified” or “to purify” refers to the removal of contaminants from a sample. For example, EPOR agonists are purified by removal of contaminating non-immunoglobulin proteins; they are also purified by the removal of immunoglobulin that does not bind EPOR. The removal of non-immunoglobulin proteins and/or the removal of immunoglobulins that do not bind EPOR results in an increase in the percent of EPOR agonist in the sample.

The term “recombinant protein” or “recombinant polypeptide” as used herein refers to a protein molecule that is expressed from a recombinant DNA molecule.

Numerous techniques that are well known in the art are used to detect antibody binding in association with the present invention. These techniques include, but not limited to RIA (radioimmunoassay), ELISA (enzyme-linked immunosorbant assays), “sandwich” immunoassays, immunoradiometric assays, gel diffusion precipitation reactions, immunodiffusion assays, SEC (size exclusion chromatography), BLI (Biolayer Interferometry), in situ immunoassays (e.g. using colloidal gold, enzyme or radioisotope labels, for example), Western blots, precipitation reactions, agglutination assays (e.g. gel agglutination assays, hemagglutination assays, etc.), complement fixation assays, immunofluorescence assays, protein A assays, and immunoelectrophoresis assays, etc.

As used herein, the term “western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies, enzyme linked antibodies, etc.

The term “antigenic determinant” as used herein refers to that portion of an antigen that contacts an antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody. A person skilled in the art will understand that the “paratope”, also referred to as the antigen-binding site, is the part of an antibody which recognizes and binds to the epitope of the antigen. Each paratope is made up of six complementarity-determining regions—three from each of the light and heavy chains. Each arm of the Y-shaped antibody has an identical paratope at the end.

The term “conformational epitope” refers to an epitope in which discontinuous amino acids that come together in three-dimensional conformation. In a conformational epitope, the points of interaction occur across amino acid residues on the protein that are separated from one another. In one embodiment, the epitope is that described in Examples of this specification.

The term “sample” as used herein is used in its broadest sense. A sample suspected of containing a nucleic acid or amino acid sequence may comprise a cell, chromosomes isolated from a cell (e.g., a spread of metaphase chromosomes), genomic DNA (in solution or bound to a solid support), RNA (in solution or bound to a solid support), cDNA (in solution or bound to a solid support) and the like. A sample suspected of containing a protein may comprise a cell, a portion of a tissue, an extract containing one or more proteins and the like.

As used herein, the term “response,” refers to the generation of a detectable signal when used in reference to an assay or other result (e.g., accumulation of reporter molecule, increase in ion concentration, accumulation of a detectable chemical product (e.g., antibody)).

As used herein, the terms “agonist” and “agonistic” refer to or describe a molecule which is capable of, directly or indirectly, initiating, activating, stimulating, or inducing one of more aspects of a response (e.g., a physiological response) or other biological activity when combined with a receptor. In a preferred embodiment, the compounds of the present invention, such as diabodies, may comprise agonists of EPO in that they mimic one or properties of EPO. As used herein, the terms “antagonist” and “antagonistic” refer to or describe a molecule which is capable of, directly or indirectly, acting against or blocking one of more aspects a physiological response or other biological activity when combined with a receptor. An antagonist is the opposite of agonist. In another preferred embodiment of the present invention, the compounds of the present invention, such as complete antibodies, may comprise antagonists of EPO in that they inhibit the actions of EPO.

As used herein, the term “antibody” or “Ab” is used in the broadest sense and specifically covers single anti-EPOR monoclonal antibodies (including agonist, antagonist, and neutralizing or blocking antibodies) and anti-EPOR antibody compositions with polyepitopic specificity. “Antibody” as used herein includes intact immunoglobulin or antibody molecules, polyclonal antibodies, multispecific antibodies (i.e., bispecific antibodies formed from at least two intact antibodies), immunoglobulin or antibody fragments (such as Fab, F(ab′)2, or Fv) and synthetic diabodies, so long as they exhibit any of the desired agonistic or antagonistic properties described herein. Various procedures known within the art may be used for the production of such antibodies directed against a specific antigen, or against derivatives, fragments, analogues, homologs or orthologs thereof.

Antibodies are typically proteins or polypeptides that exhibit binding specificity to a specific antigen. Native antibodies are usually heterotetrameric glycoproteins, composed of two identical light (L) chains and two identical heavy (H) chains. Typically, each light chain is linked to a heavy chain by one covalent disulphide bond, while the number of disulphide linkages varies between the heavy chains of different immunoglobulin isotypes. Each heavy and light chain also has regularly spaced intrachain disulphide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively.

As used herein, “antibody fragments” comprise a portion of an intact antibody, generally the antigen binding or variable region of the intact antibody. Examples of antibody fragments include Fab, Fab′, F(ab′)2, and Fv fragments, diabodies, single chain antibody molecules, and multispecific antibodies formed from antibody fragments.

The term “diabody” refers to small antibody fragments with two antigen-binding sites, wherein each antigen binding site comprises a heavy chain variable domain (VH) connected to a light chain variable domain (VL) in the same polypeptide chain (VH and VL). In a diabody, each VL domain is connected with one VH domain in a single-chain Fv (scFv) fragment by a short linker. By using a linker that is short (e.g., in a preferred embodiment of the present invention, six to fifteen amino acids or 18 Å) to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

As used herein, the term “linker” refers to a linking sequence that promotes the formation of a diabody, preferrable an 18 Å (±2 Å) amino acid linker, more preferably a linker having any amino acid combination of 5 to 15 amino acids, yet more preferably having the sequences GGGGG, IKGGGGGEV, LKVLSRGVV, ISARAGSLV, SKSRAGGEV, LKGGRGGKV, NKGGGGAKV, IKGSSRDDI, MKHAGRGGV, SKGGGGGEV, MKHAGRGGV, LECSDCSGI, yet even more preferably having the sequence GGGGG.

As used herein, the term “variable domain” describes certain portions of antibodies that differ in sequence among antibodies and are used in the binding and specificity of each particular antibody for its particular antigen. However, the variability is not usually evenly distributed through the variable domains of antibodies. It is typically concentrated in three segments called complementarity determining regions (“CDRs”), “hypervariable regions” or “hypervariable domains” both in the light chain and the heavy chain variable domains. The more highly conserved portions of the variable domains are called the framework regions (“FR”). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity.

As used herein, the term “monoclonal antibody” or “mAb” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The modifier “monoclonal” indicates the character of the antibody as being obtained from a substantially homogeneous population of antibodies and is not to be construed as requiring production of the antibody by any particular method.

It will be understood by a person skilled in the relevant art that modifications of the antibodies of the present invention are contemplated herein. The antibodies of the present invention may be modified by conjugating, tagging or labelling through methods known in the art, the antibodies of the present invention to any known diagnostic or therapeutic agent, including but not limited to cytotoxic agents (e.g., immunotoxin conjugates), prodrugs, drugs (e.g., pharmaceutically active substances) or other effector molecules which are effective in the treatment of disease as well as known reporter molecules. Such modified antibodies, also referred to as immunochemical derivatives thereof include, but are not limited to (a) labelled (e.g. radiolabelled, enzyme-labelled, fluorochrome or chemiluminescent compound) monoclonal antibodies of the present invention, preferably humanized mAbs, for diagnosing or detecting tumors and tumor spread (e.g. metastasis) using known imaging technologies; and (b) immunotoxin conjugates of the mAbs of the present invention, preferably humanized mAbs, where the mAbs of the present invention are conjugated to known cytotoxic, radioactive, radiolabelled, prodrug or drug moieties (e.g. radioimmunotherapy). It will be understood by a person skilled in the relevant art that the term “cytotoxic agent”, “cytotoxins” or “cytotoxic” as used herein generally refer to a substance that inhibits or prevents the function of cells and/or causes destruction of cells and includes, but is not limited to, radioactive isotopes, chemotherapeutic agents, and toxins such as small molecule toxins or enzymatically active toxins of bacterial, fungal, plant or animal origin, including fragments and/or variants thereof. It will also be understood by a person skilled in the relevant art that the term “prodrug” as used in this application generally refers to a precursor or derivative form of a pharmaceutically active substance that is less cytotoxic to target cells compared to the pharmaceutically active substance and is capable of being activated or converted into the more pharmaceutically active substance.

It may be understood by a person skilled in the relevant art that antibodies of the present invention can also be made by recombinant DNA methods, such as those described in U.S. Pat. No. 4,816,567, which is hereby incorporated by reference. DNA encoding the monoclonal antibodies of the invention can be readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention or can be substituted for the variable domains of one antigen-combining site of an antibody of the invention to create a chimeric bivalent antibody.

It may be understood by a person skilled in the art that “phage display” references technology for generating and selecting novel proteins that bind to a ligand, such as an antigen. Using the technique of phage display, large libraries of protein variants can be generated and rapidly sorted for those sequences that bind to a target antigen with high affinity. Methods of generating peptide libraries and screening those libraries have been disclosed in many patents (e.g., U.S. Pat. Nos. 5,723,286, 5,432,018, 5,580,717, 5,427,908 and 5,498,530). It will also be understood that antibody phage display libraries may also be created. (Smith et al., Science (1985), 228:1315; Skerra and Pluckthun, Science (1988), 240:1038). Libraries of antibodies or antigen binding polypeptides have been prepared in a number of ways including by altering a single gene by inserting random DNA sequences or by cloning a family of related genes. Methods for displaying antibodies or antigen binding fragments using phage display have been described in U.S. Pat. Nos. 5,750,373, 5,733,743, 5,837,242, 5,969,108, 6,172,197, 5,580,717, and 5,658,727. The antibody phage display library is then screened for expression of antibodies or antigen binding proteins with desired characteristics. Phage display technology has several advantages over conventional hybridoma and recombinant methods for preparing antibodies with the desired characteristics. This technology allows the development of large libraries of antibodies with diverse sequences in less time and without the use of animals. Preparation of hybridomas or preparation of humanized antibodies can easily require several months of preparation. In addition, since no immunization is required, phage antibody libraries can be generated for antigens which are toxic or have low antigenicity (Hogenboom, Immunotechniques (1988), 4:1-20). Commercially available, such as those developed by Cambridge Antibody Technology and Morphosys (Vaughan et al. (1996) Nature Biotech 14:309; Knappik et al. (1999) J. Mol. Biol. 296:57).

Generating a diverse library of antibodies or antigen binding proteins is important for isolation of high affinity antibodies. Libraries with diversification in limited CDRs have been generated using a variety of approaches. See, for e.g., Tomlinson, Nature Biotech. (2000), 18:989-994. CDR3 regions are of interest in part because they often are found to participate in antigen binding. CDR3 regions on the heavy chain vary greatly in size, sequence, and structural conformation. Others have also generated diversity by randomizing CDR regions of the variable heavy and light chains using all 20 amino acids at each position. It was thought that using all 20 amino acids would result in a large diversity of sequences of variant antibodies and increase the chance of identifying novel antibodies. (Barbas, PNAS 91:3809 (1994); Yelton, D E, J. Immunology, 155:1994 (1995); Jackson, J. R., J. Immunology, 154:3310 (1995) and Hawkins, R E, J. Mol. Biology, 226:889 (1992)).

Once binding molecules have been identified that have the desired binding and functional activity, a preferred embodiment of the present invention involves generating binding mutants, preferably one or more amino acid alterations (e.g., substitutions) are introduced in one or more of the hypervariable or CDR regions of the binding molecule. One method is the use of affinity maturation using phage display (Hawkins et al. J. Mol. Biol. 254:889-896 (1992) and Lowman et al. Biochemistry 30(45):10832-10837 (1991)). Under this technique, a number of hypervariable region sites are mutated to generate possible amino substitutions at each site. The antibody mutants thus generated are displayed and screened for their biological activity (e.g., binding affinity). A person skilled in the relevant art will understand that this process is generally referred to as affinity maturation.

It will be understood by a person skilled in the relevant art that the compositions of the present invention, including but not limited to synthetic antibodies, can be formulated into pharmaceutical compositions for administration in a manner customary for administration of such materials using standard pharmaceutical formulation chemistries and methodologies, all of which are readily available to a person skilled in the relevant art. It will also be understood by a person skilled in the relevant art that such pharmaceutical compositions may include one or more excipients, carriers, stabilizers, or other pharmaceutically inactive compounds, such as, but not limited to, wetting or emulsifying agents, pH buffering substances and the like. Pharmaceutically acceptable salts can also be included therein. A thorough discussion of pharmaceutically acceptable excipients, vehicles and auxiliary substances is available in Remington's Pharmaceutical Sciences (Mack Pub. Co. N.J. 1991), incorporated herein by reference. Such pharmaceutical compositions can be prepared as injectable or oral preparations. The antibodies of the present invention may be administered by injection, including, but not limited to, intramuscular, intravenous, subcutaneous, peritoneal, transdermic or nasal injection. The therapeutically effective doses may vary according to body weight and the timing and duration of administration will be determined by specific clinical research protocols.

The description that follows, and the embodiments described therein, are provided by way of illustration of an example, or examples, of particular embodiments of the principles and aspects of the present invention. These examples are provided for the purposes of explanation, and not of limitation, of those principles and of the invention. In the description, like parts are marked throughout the specification and the drawings with the same respective reference numerals.

The present invention is directed to the diagnosis and treatment of key disease or cancer-associated anaemia without adverse effects on disease (e.g., cancer, kidney disease) recurrence and patient survival through the selective activation or signalling of the EPOR. More preferably, embodiments of the present invention are directed to specific synthetic binding molecules, including antibodies, diabodies, etc. as a treatment for disease, including cancer associated anaemia. More preferably, the present invention is directed to methods of activating an EPOR activation site upon binding with the agonists of the present invention. The invention may be applicable to many disease states, including but not limited to cancer associated anaemia and kidney disease alone or in combination with other therapeutic agents.

Two antigen-binding sites of a synthetic agonists (e.g., antibodies or diabodies) enable the dimerization of EPOR, which is critical for its phosphorylation and activation of downstream signal pathways that lead to erythropoiesis. EPOR agonists demonstrated excellent function in promoting erythropoiesis in vitro and long-lasting activity in vivo. These EPOR agonists may represent a novel therapeutic option for patients with chronic kidney disease or treatment related anaemia.

### Examples

### Production, Preparation and Characterization of EPOR Binding Molecules

To select EPOR agonists, a diabody library was constructed by modifying a phagemid vector encoding a human framework scFv6. In a preferred embodiment, the C-terminus of the variable light (VL) domain may be linked to the N-terminus of the heavy chain variable (VH) domain by a linker, preferably a Gly5 linker (e.g., GGGGG), producing a diabody linked to the M13 gene-3 minor coat protein via a modified IgG hinge sequence. In a preferred embodiment, four of the six CDRs—the three heavy chain CDRs and CDR-L3—were diversified as described previously for library F1, although with a reduced length diversity for CDR-H3. This library contained 4.2×10⁹ unique clones and was used for selections as previously described¹.

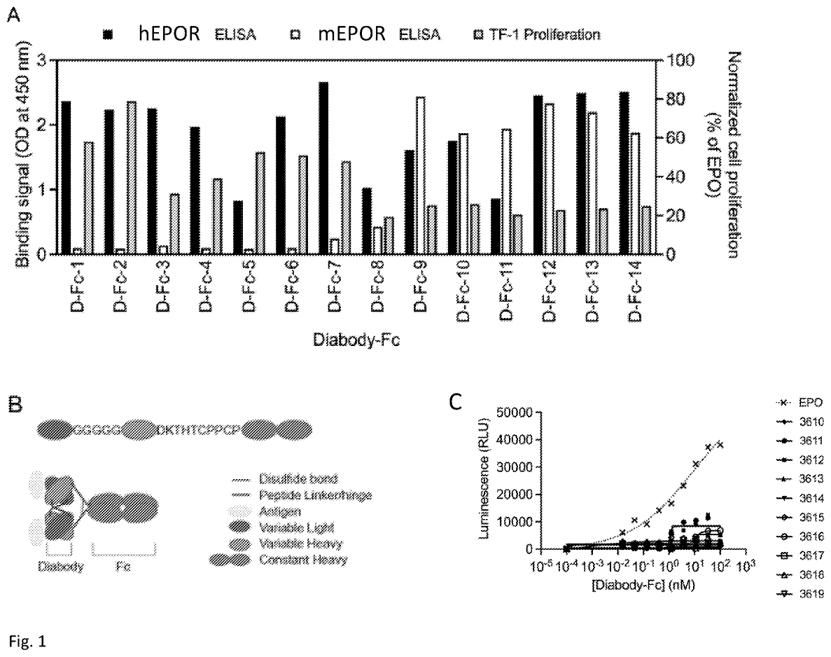

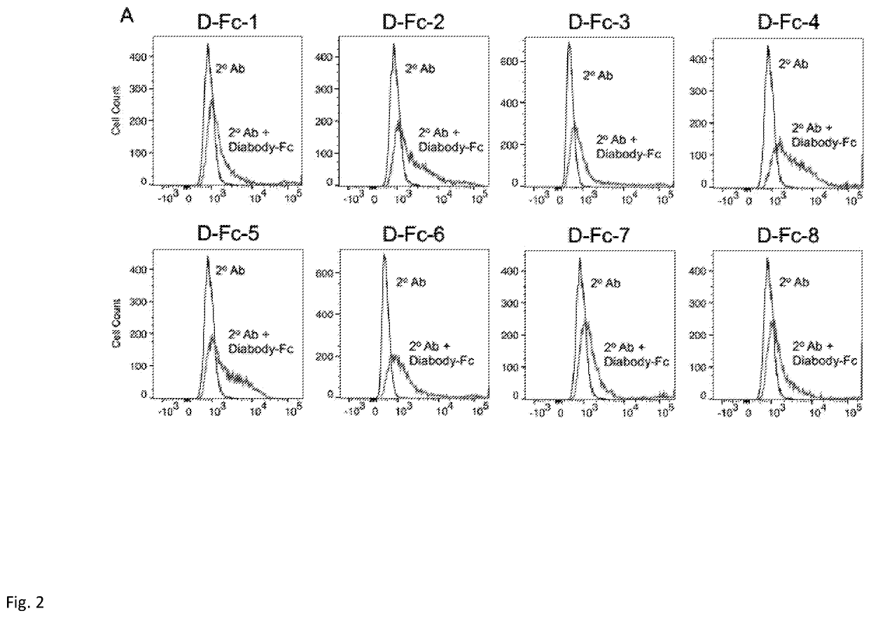

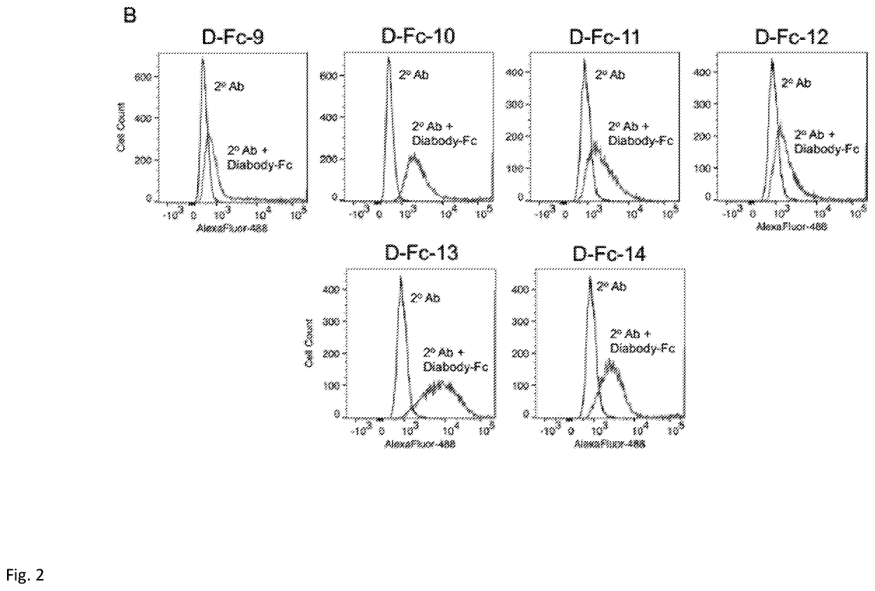

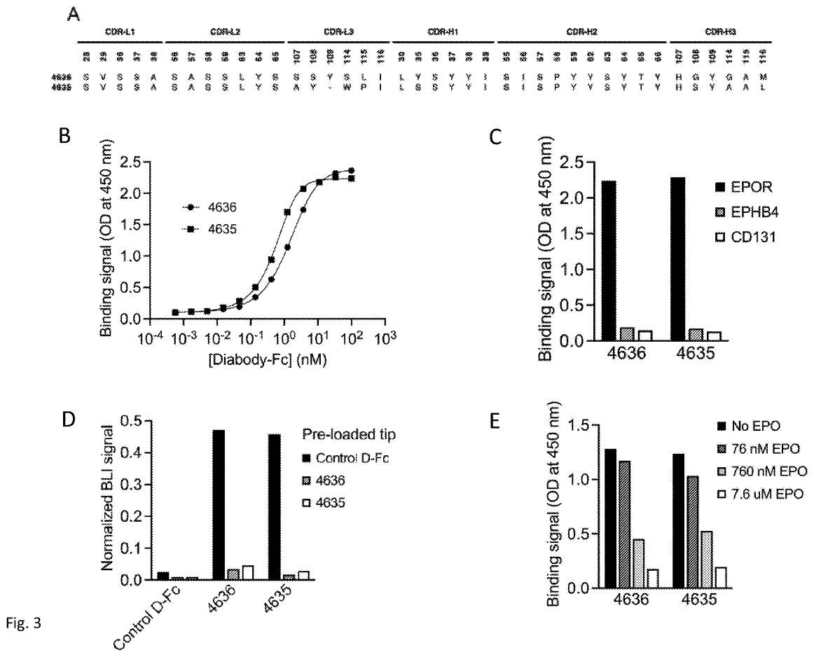

Using the diabody-phage library as described herein, binding selections with the recombinant extracellular domain (“ECD”) of human EPOR (“hEPOR-ECD”) were performed. Phage-Ab that bound specifically to antigen (e.g., ECD) and not control proteins by ELISA were sequenced by PCR amplification, and unique sequence clones were identified. Sequencing of individual binding clones identified 14 unique clones that bind human and murine EPOR in vitro as purified diabody-Fc (fragment crystallizable region) proteins (“D-Fcs”) (see FIGS. 1A and 1B). The attachment of the Fc region is also thought to prolong the half-life of the diabodies in serum. These 14 D-Fcs also bound to cell-surface expressed EPOR as demonstrated by flow cytometry analysis using human erythroid TF-1 cells (FIGS. 2A and 2B). However, of the 14 D-Fc, only 7 (D-Fc-1 to D-Fc-7) stimulated proliferation of TF-1 cells as would be expected of EPOR activation (see FIG. 1A). Interestingly, all D-Fcs with significant TF-1 agonist activity (>40% growth) generally had little cross-reactivity to mouse EPOR (see FIG. 1A). In addition, we tested the agonistic properties of several naïve IgG clones that were derived from library F selections using human EPOR as the immobilized target as previously described¹. None of these IgGs had any significant effect on cell proliferation (FIG. 1C). This is in stark contrast to previous work describing EPOR IgG agonists and clearly distinguishes the current embodiments as novel and non-obvious². The two most potent clones (D-Fc-1 and D-Fc-2) were chosen for further characterization and, hereafter, referred to as 4636 and 4635, respectively (see, for example, FIG. 3A). These 2 D-Fc constructs showed significant homology in their heavy-chain CDR sequences, suggesting that they likely bind to the EPOR in a similar manner.

To characterize the mechanism of action of 4636 and 4635, titration ELISAs (enzyme linked immunosorbent assays) were performed. Binding to immobilized hEPOR was plotted versus diabody concentration and the graphs were used to calculate EC50 values of 1.5 nM and 0.56 nM for 4636 and 4635, respectively (see FIG. 3B). Single-concentration ELISAs were also used to examine specificity of 4636 and 4635. Two receptors previously shown to bind to EPO are EPHB4 and CD131. However, neither diabody-Fcs demonstrate any significant binding to those receptors (see FIG. 3C). An epitope binning assay was then performed using competition Biolayer Interferometry (“BLI”) to determine whether 4636 and 4635 bound to common epitopes (see FIG. 3D). Briefly, EPOR was immobilized and used to capture the indicated diabody-Fc in solution-phase at a saturating concentration (pre-loaded tip). After excess diabody-Fcs were washed off, a control D-Fc, 4636, or 4635 was loaded again in solution phase. If an epitope on EPOR was blocked by the saturating D-Fc pre-load, there would be no D-Fc binding in this second round. It was observed that both 4636 and 4635 blocked each other, indicating that these two diabody-Fcs shared a similar epitope (see FIG. 3D). Competition ELISAs were used to determine if this epitope also overlapped with EPO. Both 4636 and 4635 binding to immobilized EPOR was blocked with increasing concentrations of EPO (see FIG. 3E).

### Functional Activity of EPOR Binding Molecules

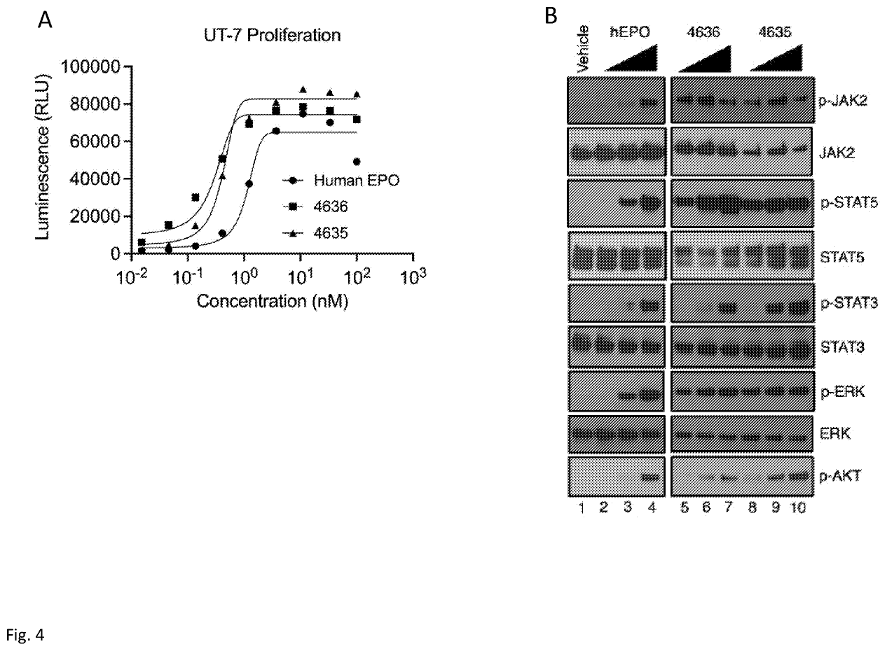

Activation of EPOR triggers the proliferation of human erythroid cell lines. Thus, the ability of 4636 and 4635 to stimulate proliferation of the erythroid UT7/EPO cell line was tested. Using a bioluminescence-based cell proliferation assay, both 4636 and 4635 were potent inducers of UT7/EPO cell growth. In fact, they were both more effective than EPO itself (see FIG. 4A). Western blotting was then used to examine the biochemical effects of 4636 and 4635 in UT7 cells. Functional activity of erythropoiesis-stimulating agents requires dimerization of EPOR that initiates a sequential cascade of protein phosphorylation events, including the phosphorylation of JAK2, STAT3/5, ERK, and AKT. These modifications were monitored in UT-7/EPO cells in response to 4636 and 4635 (see FIG. 4B). UT-7/EPO cell line is an EPO-dependent human erythroid cell line that endogenously expresses hEPOR. It has normal EPOR expression unlike TF-1 cells3. Both 4636 and 4635 induced phosphorylation of EPOR downstream effectors very similarly to EPO (see FIG. 4B). EPO induced phosphorylation of canonical targets JAK2, STAT3, STAT5, ERK and AKT in a dose dependent fashion. Importantly, 4636 and 4635 both induced phosphorylation of all 5 signalling molecules in a manner very similar to EPO.

### Affinity Maturation of EPOR Binding Molecules

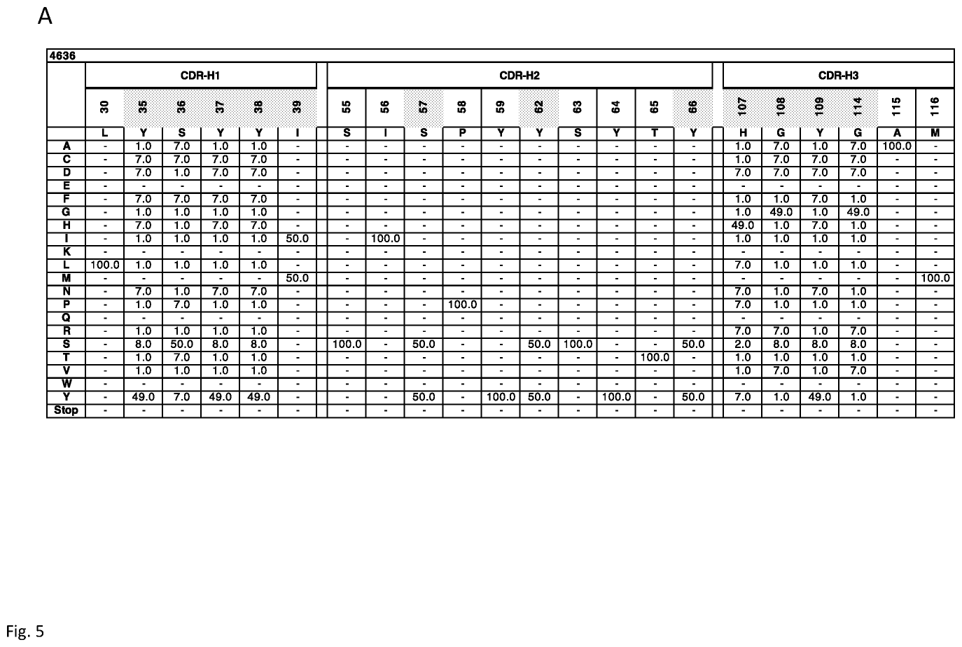

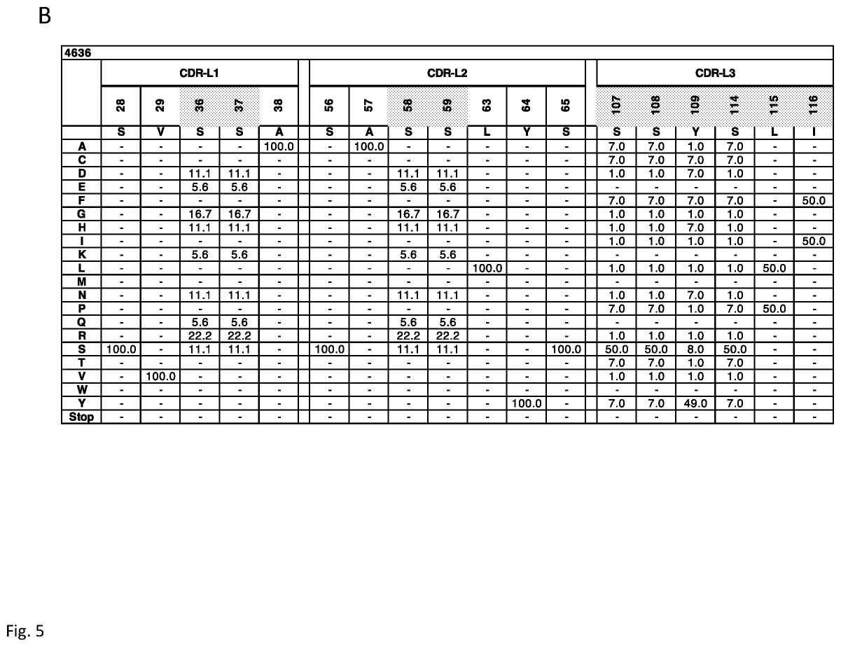

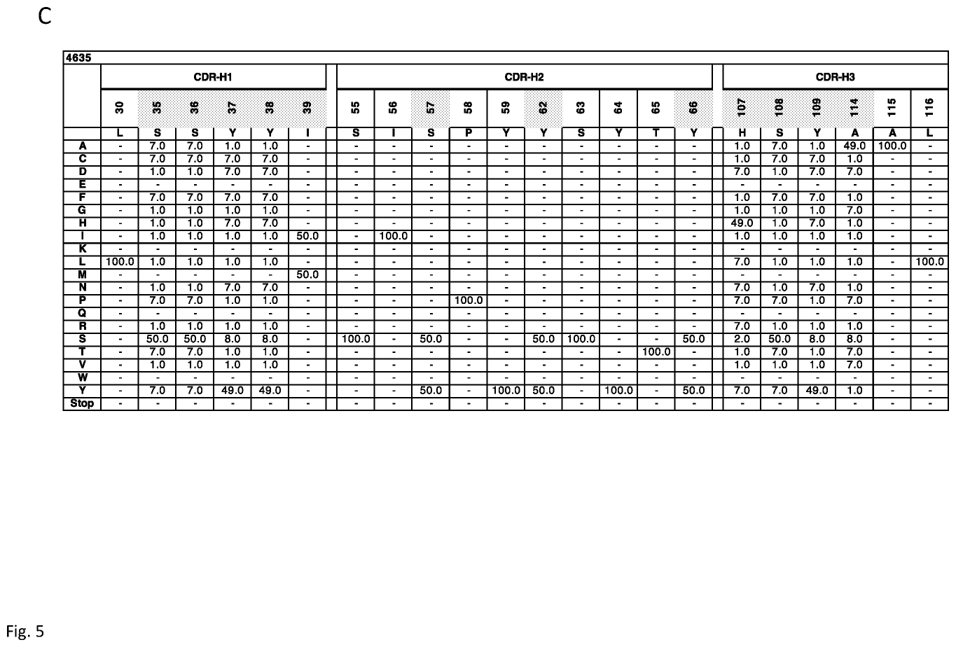

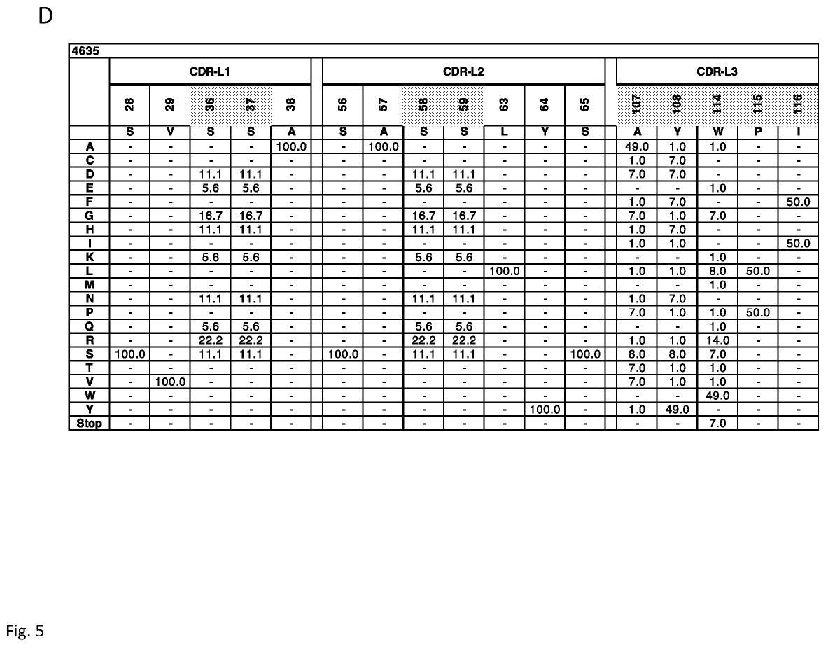

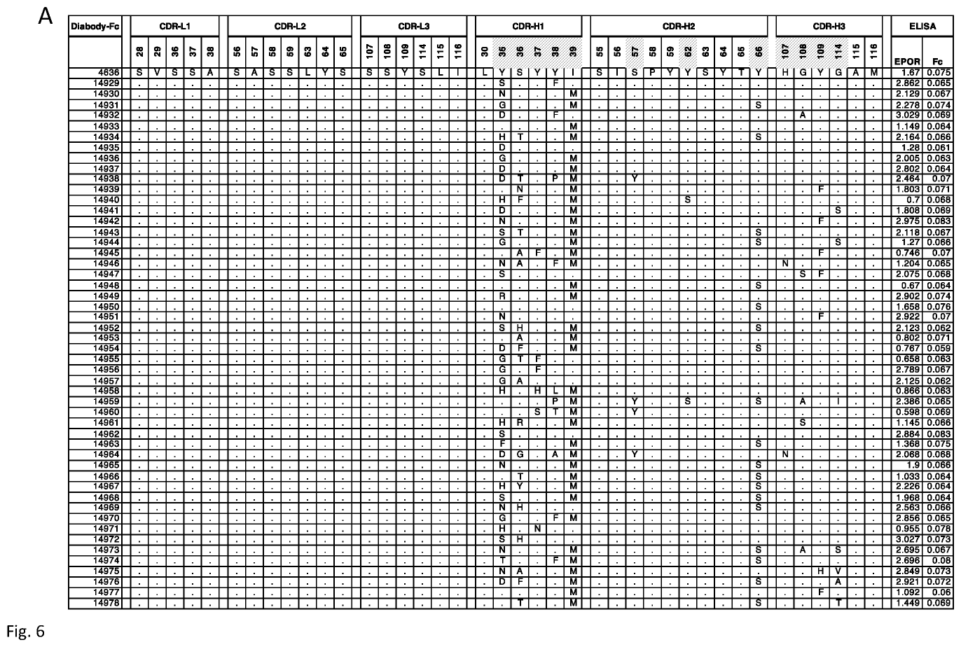

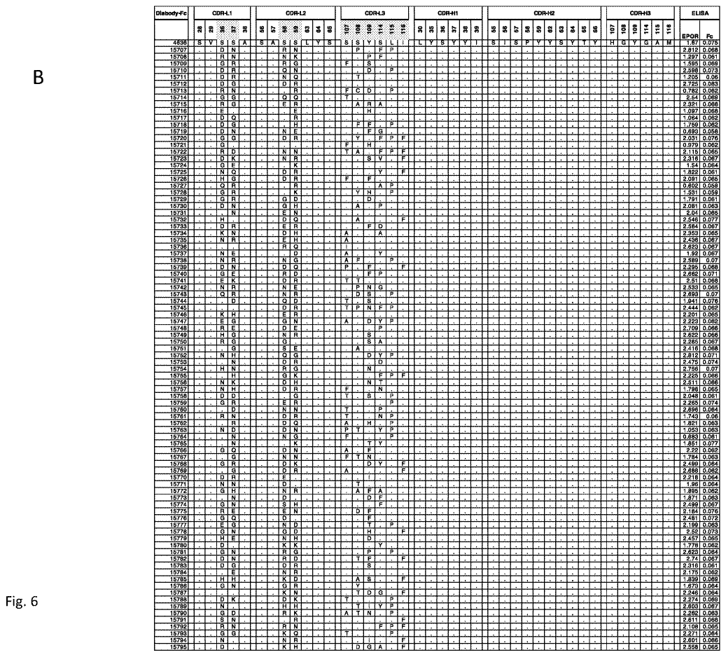

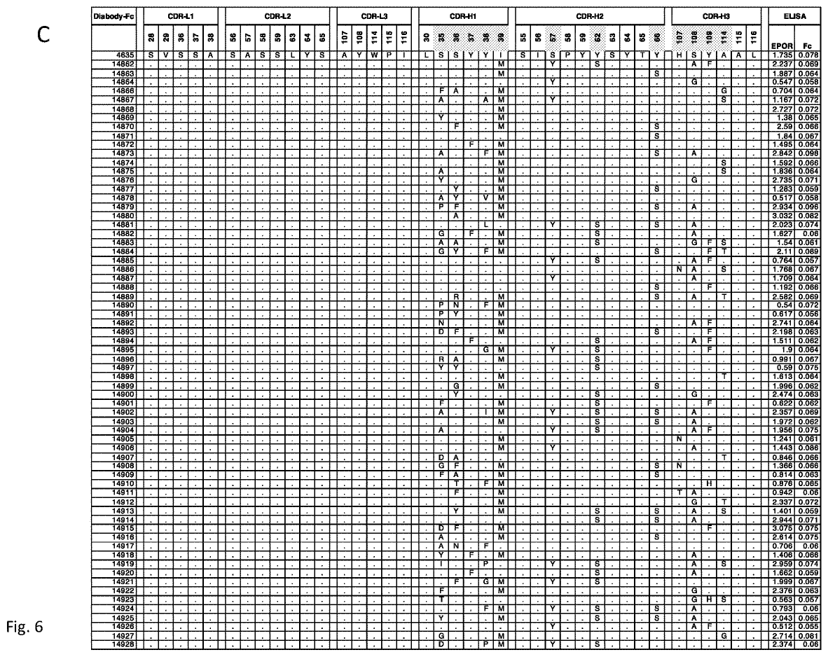

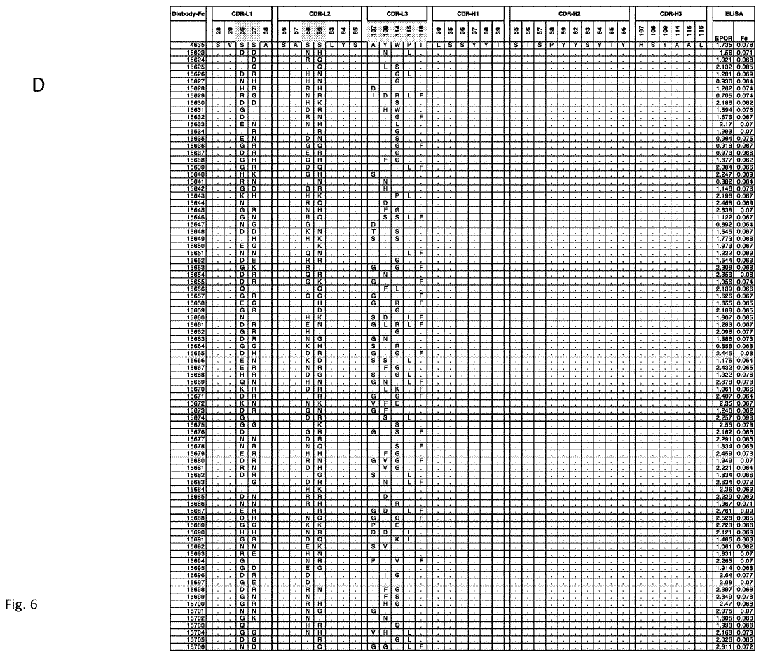

Phage antibody display affinity maturation libraries were developed to engineer further embodiments of the present invention in accordance with the strategy noted herein. Four libraries were made—one heavy chain (HC) and one light chain (LC) library for each 4636 and 4635. The exact positions within the complementarity-determining regions (CDRs) and the randomization schemes are shown in FIGS. 5A to 5D. Following 5 rounds of selections against immobilized hEPOR-ECD, sequencing of binding phage clones revealed the unique sequences of the affinity maturation clones (FIGS. 6A to 6D). Phage ELISAs were performed to confirm target binding compared to an immunobilized Fc control (see FIGS. 6A to 6D). Plates were coated with EPOR-His or Fc before phage clones expressing each diabody were added to the wells and incubated for an additional hour at room temperature. Samples were washed and then incubated with an anti-M13 secondary antibody. TMB substrate was used for colour development. OD450 was measured using a plate reader. All phage clones tested showed significant and specific binding to EPOR-His. The frequency and distribution of amino acids in the randomized positions are shown schematically in FIGS. 6A to 6D. This analysis showed that the LC positions either have a strong preference for the parental amino acid (CDR-L3) or were under little selection pressure given the random distribution of amino acids (CDR-L1 and CDR-L2). Similarly, the HC positions also tended to have a strong preference for the parental amino acid except for position 39 in CDR-H1, which favoured an isoleucine to methionine substitution.

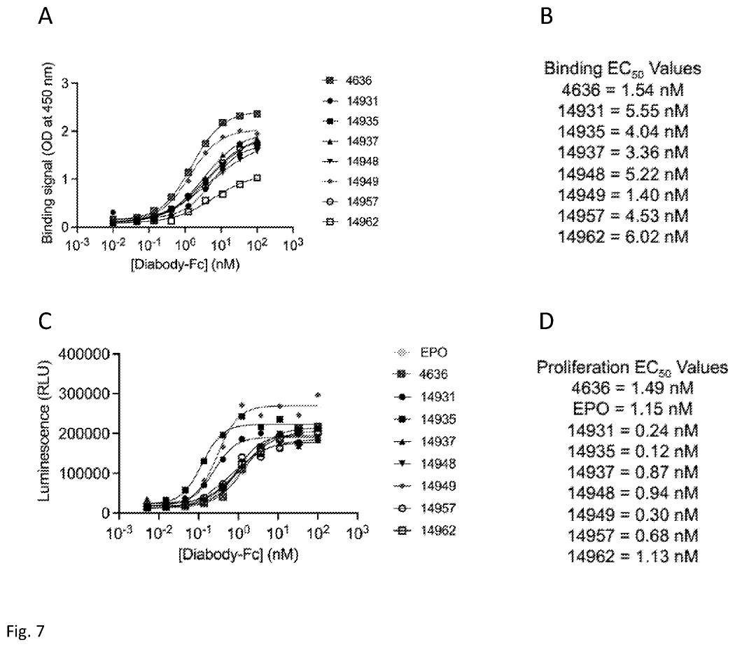

Based on the amino acid distributions, 7 clones from the HC selections were picked for further screening, which maintained the parental LC positions. These 7 clones from the HC selections all had methionine at position 39 and various amino acids at position 30 of CDR-H1. These 7 clones were cloned in D-Fc format and purified as proteins. Titration ELISAs found that a preferred embodiment, affinity maturation derived clone 14949 D-Fc (see FIG. 6A), had a very similar EC50 as 4636 (FIGS. 7A and 7B). Cell proliferation assays performed using UT7/EPO cells also found that clone 14949 was more effective (higher maximum effect) and approximately 5× more potent (lower EC50) than parental 4636 and EPO (FIGS. 7C and 7D). Together, the functional data showed that 4636, 4635, and their affinity maturation derived clones bound with high affinity to the hEPOR but not to other receptors that interact with EPO, and moreover, they competed with each other and with EPO for receptor engagement. One 4636 affinity maturation derived clone or affinity matured clone 14949 exhibited preferable or desired functional properties.

The EPOR Configuration in Complex with Affinity Maturation Derived Clone 14949 Fab

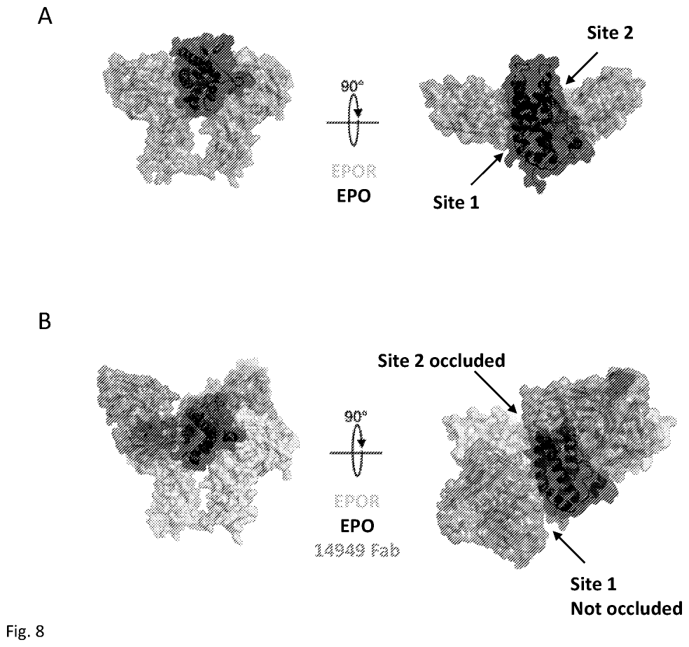

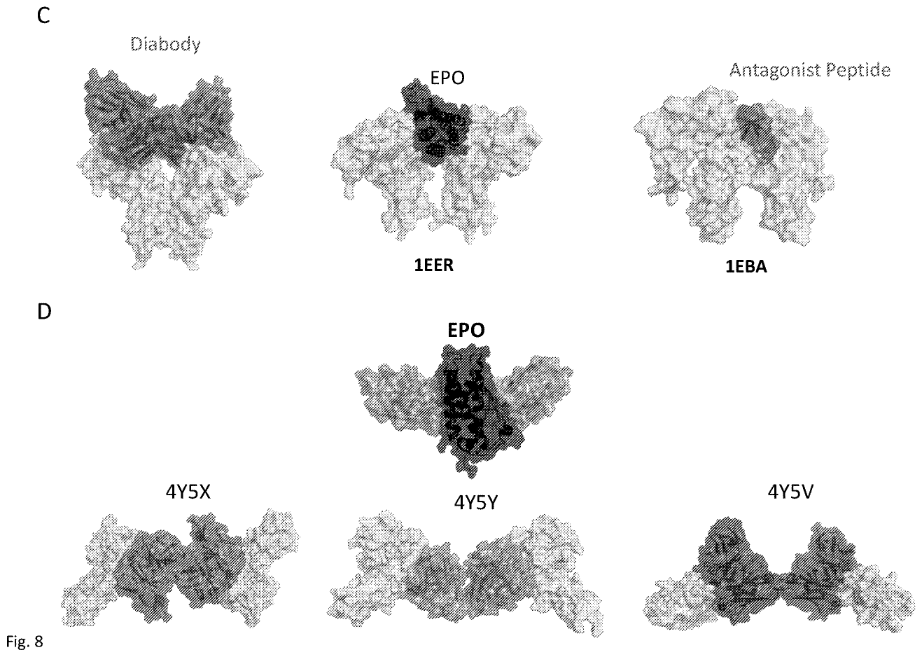

To explore the structural basis for the embodiments of the present invention binding to EPOR, one construct, identified as preferred embodiment affinity maturation derived clone 14949 of the present invention, was cloned, expressed, and purified as a Fab and crystalized in complex with hEPOR-ECD (FIGS. 8A and 8B). Human EPO binds to its cognate receptor via two sites—a high affinity site 1 located on one receptor and a low affinity site 2 of a neighbouring receptor. Binding at these sites induces the formation of an asymmetric dimer signalling complex (FIG. 8A). Using this as a basis to understand how 14949 activates EPOR signalling, the 14949-Fab/EPOR structure was refined to accommodate for the 18 Å linker in its lowest energetic state. EPO was then superimposed to reveal how high affinity site 1 remained accessible while site 2 was occluded (FIG. 8B). This explains earlier data showing that EPO competes for binding with current embodiments of the present invention (see, for example, FIG. 3E).

As shown in FIG. 8C, the model of the preferred embodiment is compared to known EPOR structures. This refined model bears striking resemblance to both the EPO-EPOR model as well as an asymmetric dimer formed using an antagonist peptide (Ref. 4). Thus, dimerization per se may not explain agonistic properties of the preferred embodiment 14949 and suggests a separate and distinct mechanism of action.

As shown in FIG. 8D, the asymmetric dimer formed by 14949 is distinct from previously developed diabodies that activate EPOR⁵. All previous diabodies induce dimerization of EPOR in a manner distinct from that of EPO. The structural resemblance of the modelled ternary 14949-EPOR complex is unique compared to prior art.

The 14949 Fab Paratope and hEPOR Epitope

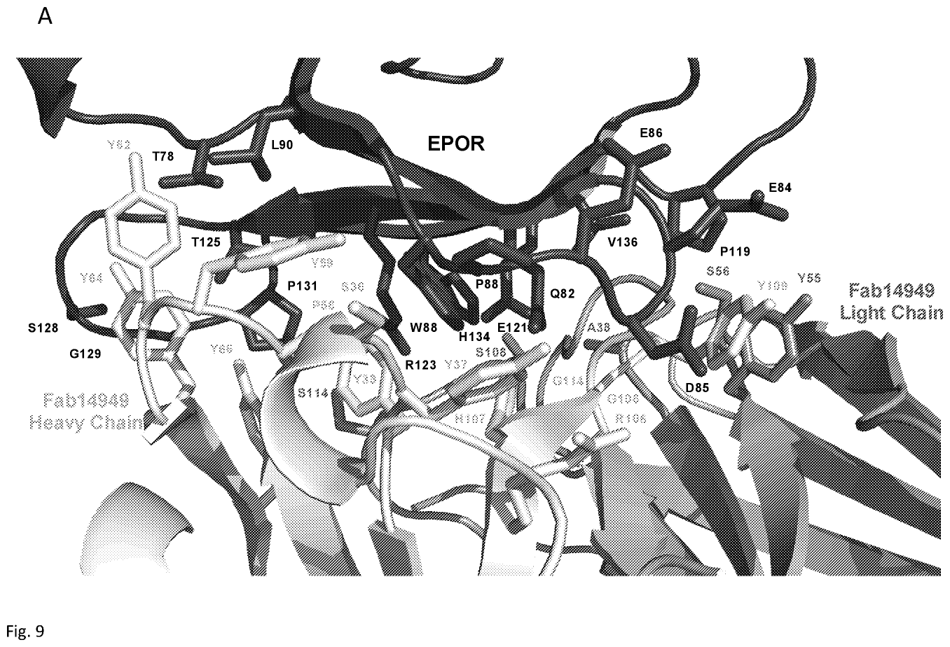

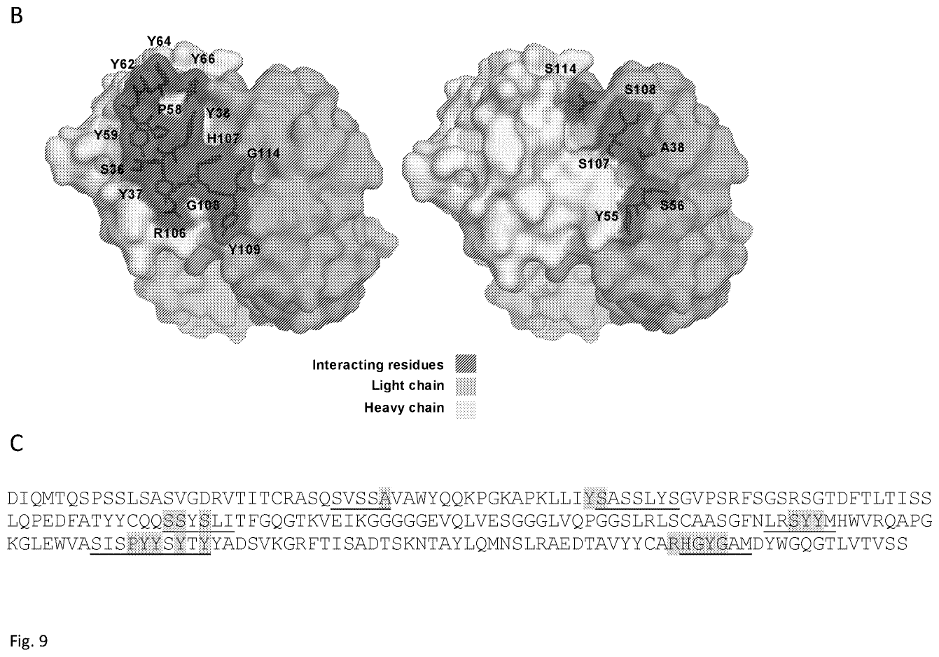

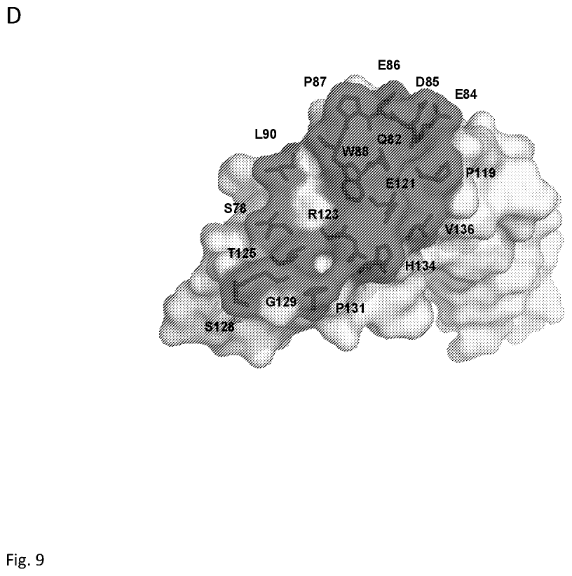

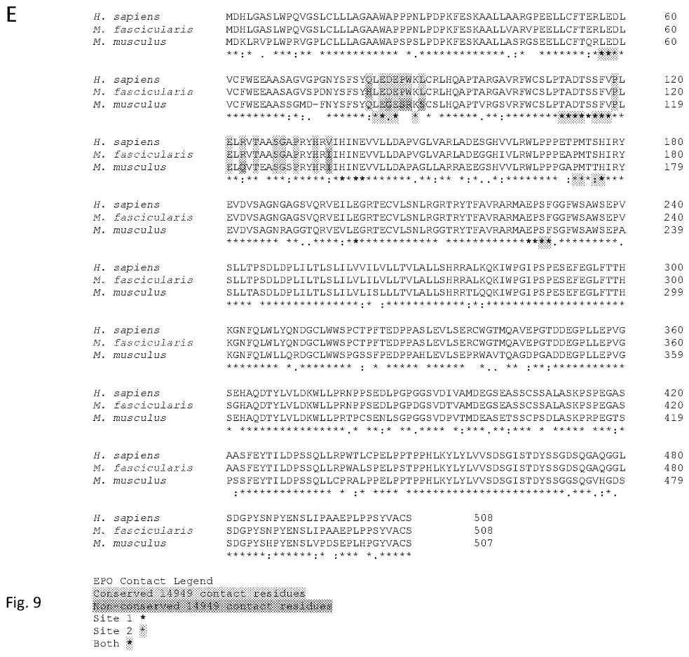

A closer examination of the 14949 Fab-EPOR complex reveals the precise residues that comprise the paratope in the Fab and the hEPOR epitope (FIG. 9A). In the paratope, several contacts are made by both VL and VH domains (FIGS. 9B and 9C). These positions all lie within CDRs. In the VL domain, these interacting CDR positions of 14949 are: Ala38 in CDR-L1; Tyr55 and Ser56 in CDR-L2; and Ser107, Ser108, and Ser114 in CDR-L3. In the VH domain, interacting residues include: Ser36, Tyr37, and Tyr38 in CDR-H1; Pro58, Tyr61, Tyr62, Tyr64, and Tyr66 in CDR-H2; and Arg106, His107, Gly108, Tyr109, and Gly113 in CDR-H3.

The hEPOR residues that comprise the epitope (FIGS. 9D and E) include Gln82, Glu84, Asp85, Glu86, Pro87, Trp88, Leu90, Pro119, Glu121, Arg123, Thr125, Ser128, Gly129, Pro131, His134, and Val136. As shown previously (FIGS. 3E and 8B), site 2 positions encompassing Leu83 to Lys89 overlap with 14949 Fab binding. Also, all except two of these binding residues (Gln82 and Val136) are identical in M. fascicularis EPOR. In contrast, there are several non-conserved substitutions found in the murine EPOR namely at positions 85, 87, 88, 90, and 123. This may explain the lack of binding and of current embodiments to murine EPOR (FIG. 1A).

### Comparison of the Epitope Recognized by 14949 Fab to Prior Art

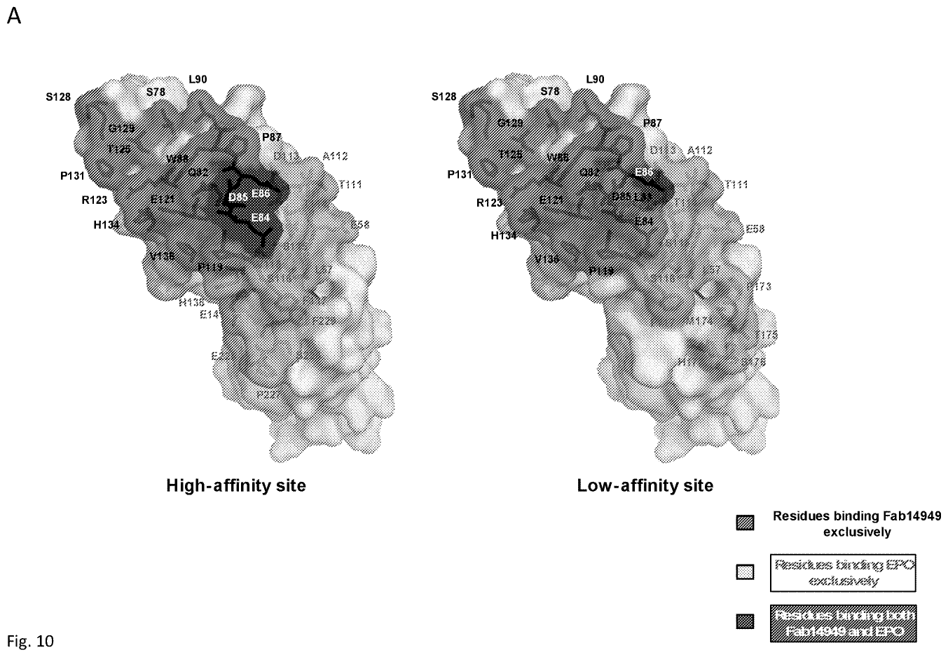

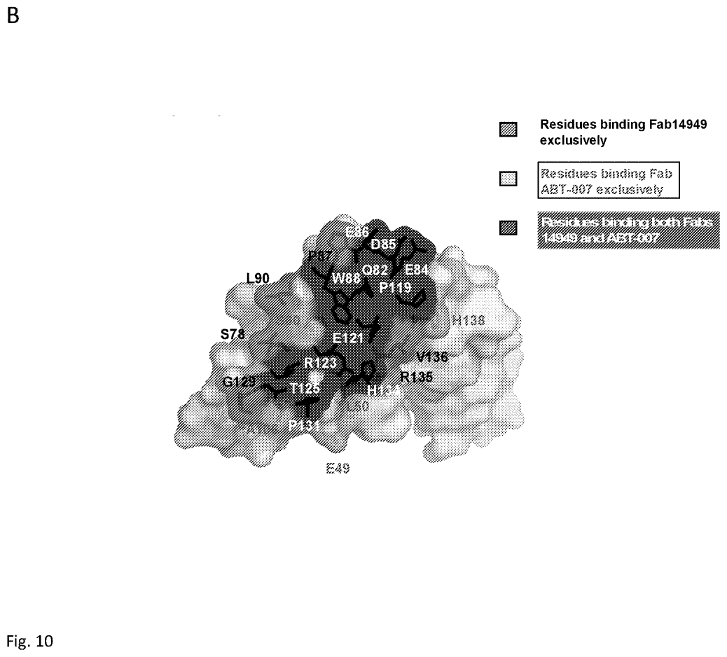

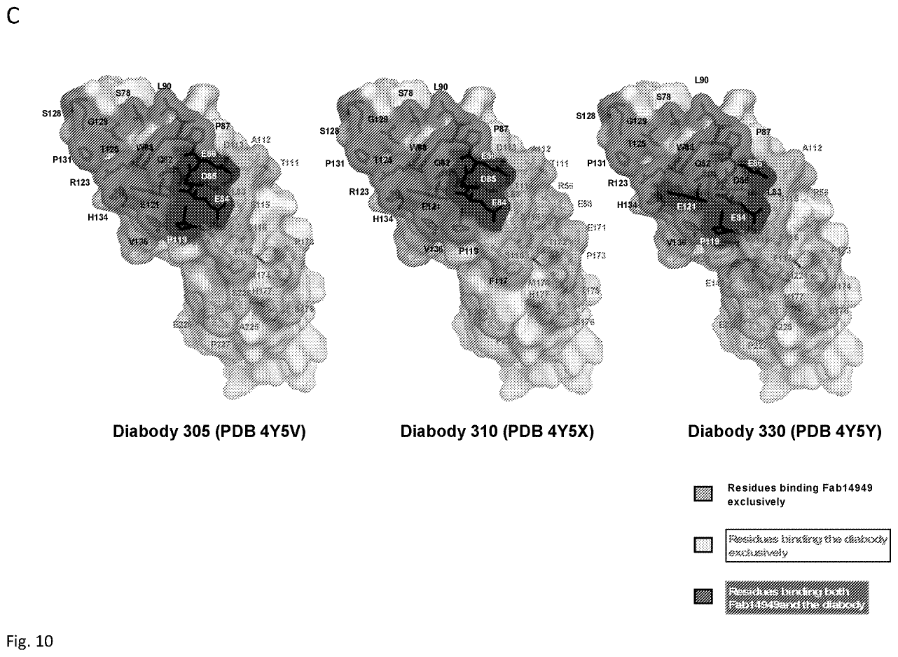

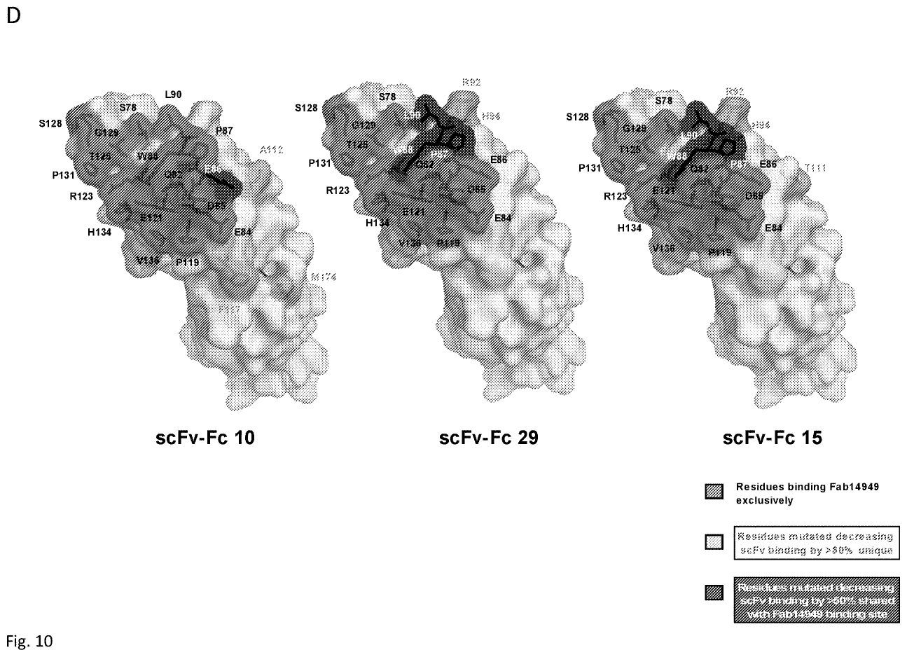

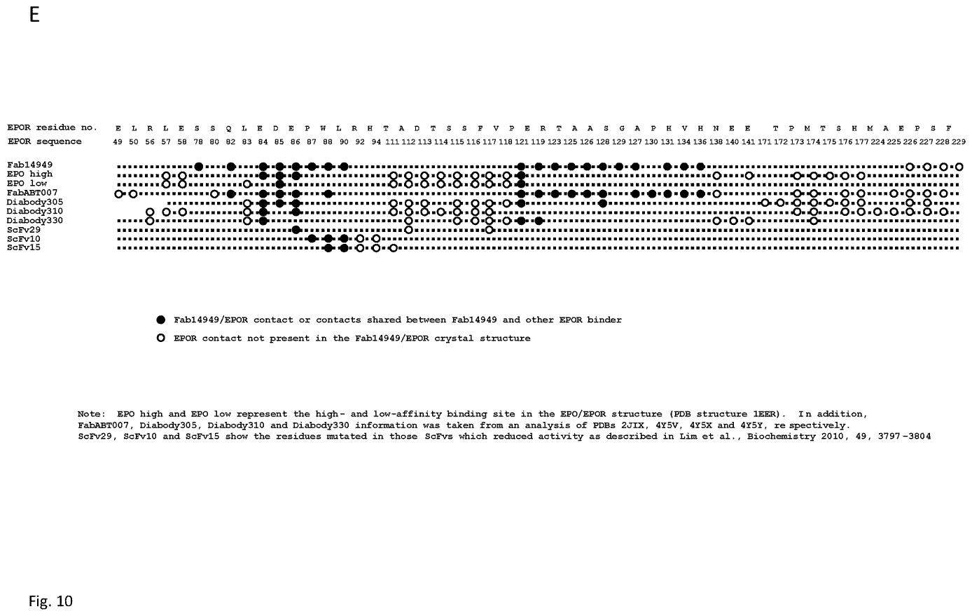

The novelty and non-obvious nature of the current invention can best be highlighted by directly comparing the epitope recognized by 14949 Fab to all prior art starting with the natural binding site for hEPO (Ref. 7) (FIG. 10A). hEPO binds to its cognate receptor through high- and low-affinity binding sites. Two residues (Glu84, and Asp85) in the high affinity site 1 are also binding residues to the 14949 Fab. Glu86 participates in both site 1 and site 2 binding and also interacts with 14949 Fab. The vast majority of the 14949 Fab epitope is unique compared to hEPO, underscoring how its mechanism of activation is non-natural.

Next, the 14949 Fab binding epitope will be compared to the previously identified antibody hEPOR agonist, ABT-007 (Ref. 2) (FIG. 10B). There is greater overlap between these two interfaces than the hEPO interface (FIG. 10A). Eleven residues on hEPOR participate in both binding to 14949 Fab and ABT-007. Only 3 residues specifically bind to 14949 Fab and do not engage ABT-007—Ser87, Pro87, and Leu90 (FIG. 10B, mid-grey residues). However, the mechanism of ABT-007 target engagement is significantly different than 14949 Fab since its epitope is much larger than that of 14949 Fab. This is not fully shown in this structure but is summarized in FIG. 10E.

Previous studies have identified three diabodies—305 (4Y5V), 310 (4Y5X), and 330 (4Y5Y)—that had modest agonistic activity (Ref. 5). Thus, a comparison was made between those epitopes and that of 14949 Fab (FIG. 10C). Out of 22 residues in the diabody-305 epitope, only 3 are shared by 14949—Glu84, Asp85, and Glu86 (FIG. 10C, left panel). For diabody-310, 19 residues formed that epitope but only 2—Glu84 and Asp85—are in common with 14949 Fab (FIG. 10C, middle panel). Similarly, only 3 out of 14 epitope residues for diabody-330 are also found in the 14949 Fab epitope (FIG. 10C, right panel). This discrepancy between 14949 Fab binding and these past diabodies is not surprising given that these past diabodies were all derived first from the IgG modality whereas 14949 was engineered from a diabody library. There is very little similarity between the underlying molecular mechanisms of 14949-mediated activation of the EPOR and the manner in which these diabodies function.

Comparisons to prior art of scFv-Fc proteins to the current invention further reveals how the present embodiments are unique (Ref. 8) (FIG. 10D). There were 3 scFv-Fcs that were identified to have high agonistic activity towards hEPOR-scFv-Fc-10, -29, and -15. Although the structures of these molecules in complex with hEPOR were never resolved, mutagenesis studies were used to map the interface. The epitope was defined as any hEPOR residue that, when mutated to Alanine, resulted in a >50% reduction in binding (Ref. 8). These positions were compared to the 14949 Fab epitope (FIGS. 9D and 9E). For the most part, the interacting residues for all 3 scFv-Fc proteins were distinct from those of 14949 Fab. In fact, scFv-Fc-10, -29, and -15 only had 3, 1, and 2 overlapping contact residues, respectively (FIGS. 10D and 10E).

Overall, comparisons to the 14949 Fab to all prior art is summarized in FIG. 10E. It demonstrates that 14949 VL and VH domains contact its hEPOR target in a very distinct fashion that has never been previously described. It is non-natural as it is also not observed for hEPO. Thus, the mechanism of action of the current embodiment is novel and non-obvious.

### Characterization of Different Diabody Modalities

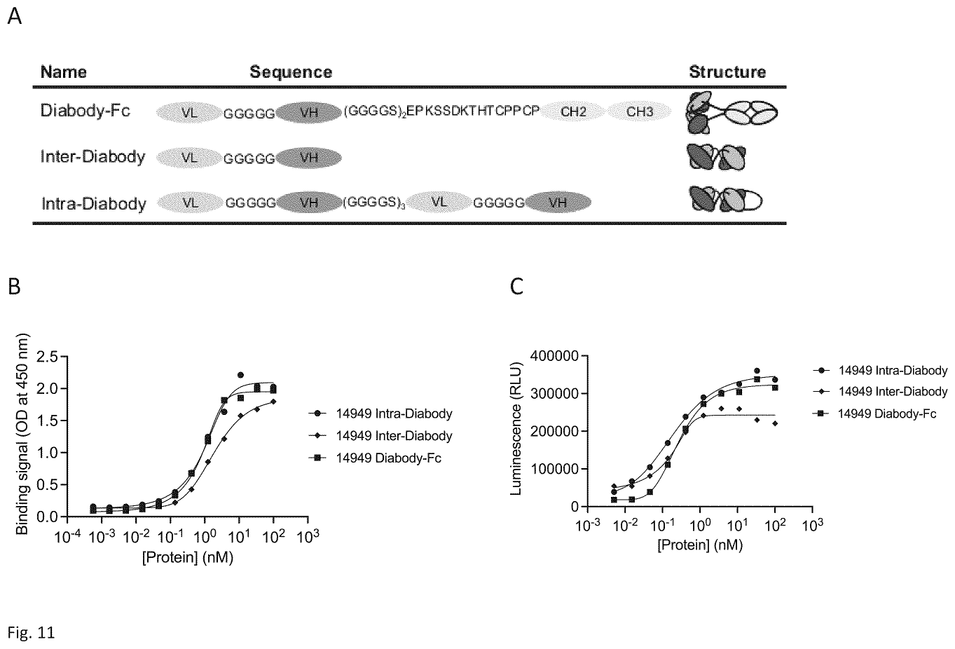

Additional experiments demonstrate how the embodiments of the invention may be functionally active when expressed in different modalities (e.g., as shown in FIG. 11A). Each format of the EPOR agonists has its unique characteristic. For example, diabody-Fc has prolonged serum half-life due to the attachment of Fc fragment; intermolecular diabody (or diabody) is significantly smaller and more similar in size to natural ligand EPO; and intramolecular diabody with a (GGGGS)3 linker allows the formation of bivalent dimer within one fragment. The Fc modality had been tested earlier and the latter modalities were then tested for binding to human EPOR by titration ELISAs and induction of UT7/EPO cell proliferation (FIGS. 11B and 11C). Whether the embodiments of the current invention are expressed as a diabody-Fc, inter-diabody, or a covalently linked intra-diabody, they all have similar target binding and agonistic effects.

### Additional Affinity Maturation of 14949

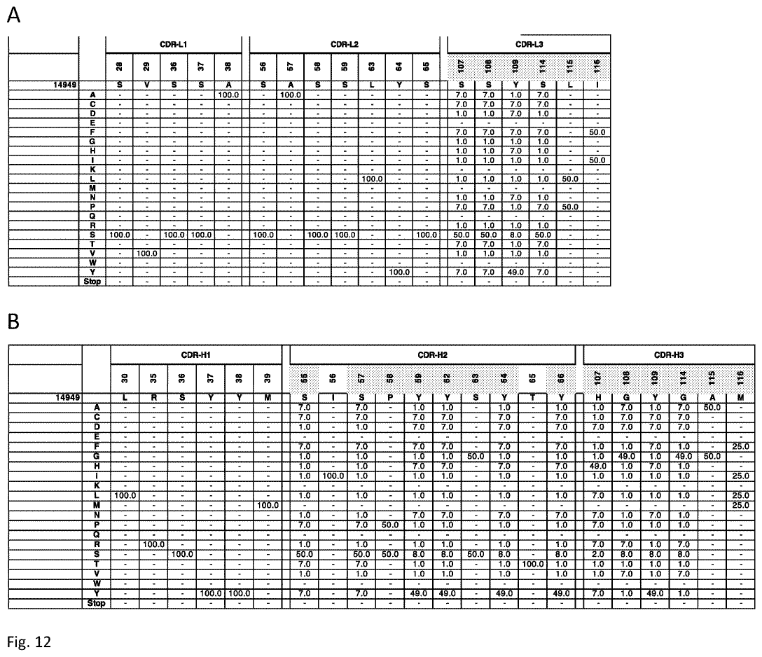

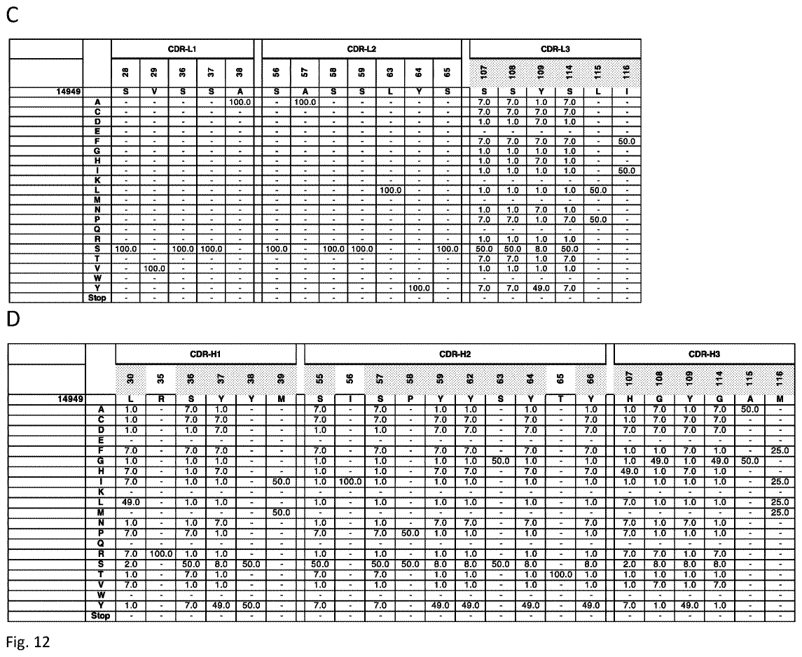

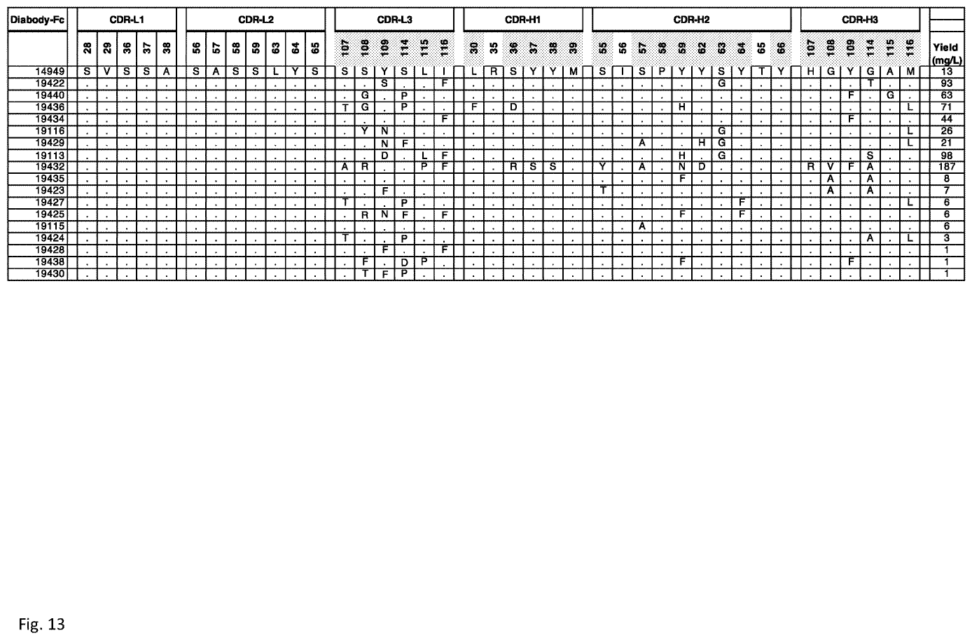

Phage antibody display affinity maturation libraries were developed to engineer further embodiments of 14949 in accordance with the strategy noted herein. Two sub-libraries were made—AP229 and AP230. Each sub-library consisted of one HC and one LC library. The exact positions within the complementarity-determining regions (CDRs) and the randomization schemes are shown in FIGS. 12A to 12D. Following 5 rounds of selections against immobilized hEPOR-ECD, binding phage clones were then used in phage ELISAs where the antigen was first pre-saturated with 100 nM 14949 D-Fc protein. Excess 14949 D-Fc protein was washed off with 5× wash with 1×PBS and the phage ELISA procedure continued using anti-M13 secondary antibody and TMB substrate for colour development. The sequences of phage clones that showed significantly reduced binding by 14949 are shown in FIG. 13, which were then cloned and expressed as diabody-Fc fusion proteins. The transient production yield (mg/L) using HEK293expi cells are also shown (FIG. 13). The proteins with a higher yield were selected for further experiments. Despite its high yield, 19432 was not progressed further because of the introduction of an N-glycosylation site in CDR-H2.

### Characterization of 14949-Family of Clones

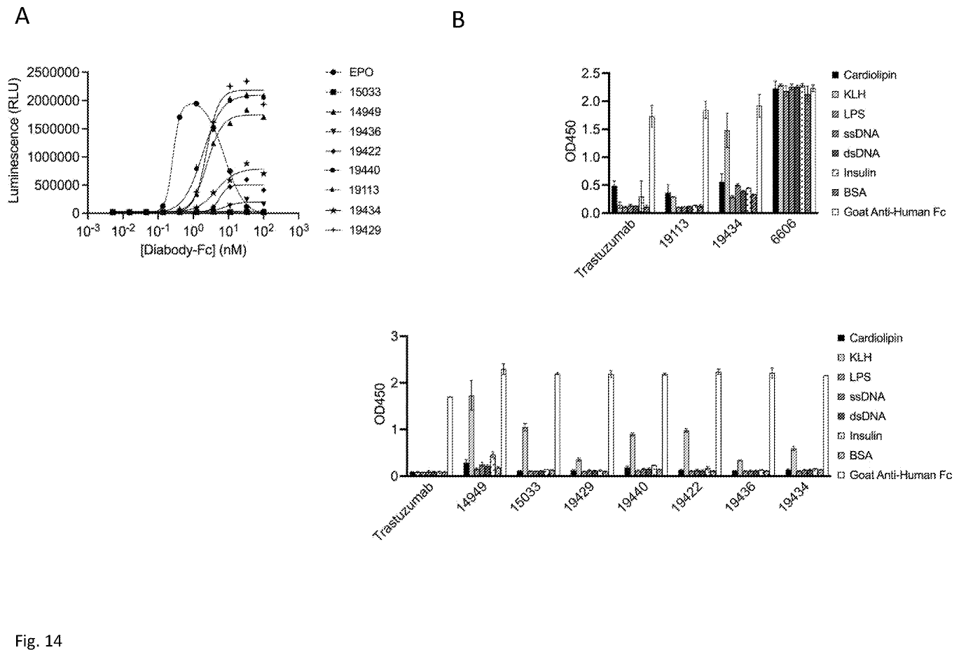

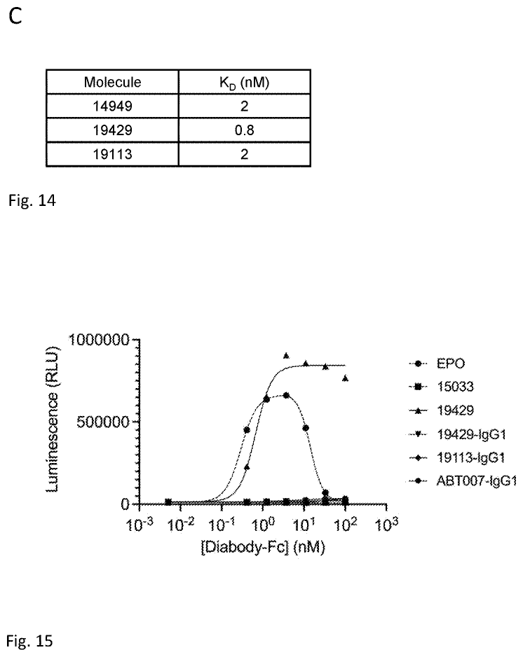

Functional analysis was performed on the high-yield clones shown in FIG. 13. Two molecules—19113 and 19429—outperformed the parental 14949 diabody-Fc, with a higher maximal proliferative response in UT7/EPO cells (FIG. 14A). A non-specificity ELISA was performed using a representative panel of antigens (FIG. 14B). All daughter clones except for 19434 showed superior specificity with much less binding to these unrelated antigens. In particular, 19113 and 19429 were the best with even minimal binding to KLH (FIG. 14B). Trastuzumab was used as a control to determine how a clinical-grade molecule would perform in this assay. 15033 was a negative control and 6606 is a highly cross-reactive control that has high background binding to a variety of antigens. The binding affinity of 19113 and 19429 to hEPOR was then measured using BLI (FIG. 14C). Compared to the parental 14949, 19113 and 19429 diabody-Fc proteins bound with similar dissociation constants, indicating that its improved hEPOR activation is likely related to receptor geometry and not as much binding strength. This is consistent with recent findings that agonism is not always correlated with affinity (Ref. 9).

### Modality is Crucial for Agonism of Lead Molecules

The lead molecules 19113 and 19429 diabody-Fc molecules were cloned and expressed as IgG1 proteins and functionally tested in a UT7/EPO cell proliferation assay (FIG. 15). Unlike the 19429 D-Fc format, as IgG1s, neither 19113 and 19429 had much agonistic activity. The previously identified ABT-007 agonistic IgG also did not exhibit much functional agonism. This experiment clearly shows that our VL/VH domains derived from our engineering do not function as classical antibodies and adds evidence to the claim that the current embodiments are novel agonist that exert their effects through a non-obvious mechanism of action.

### Additional Variants of 19429

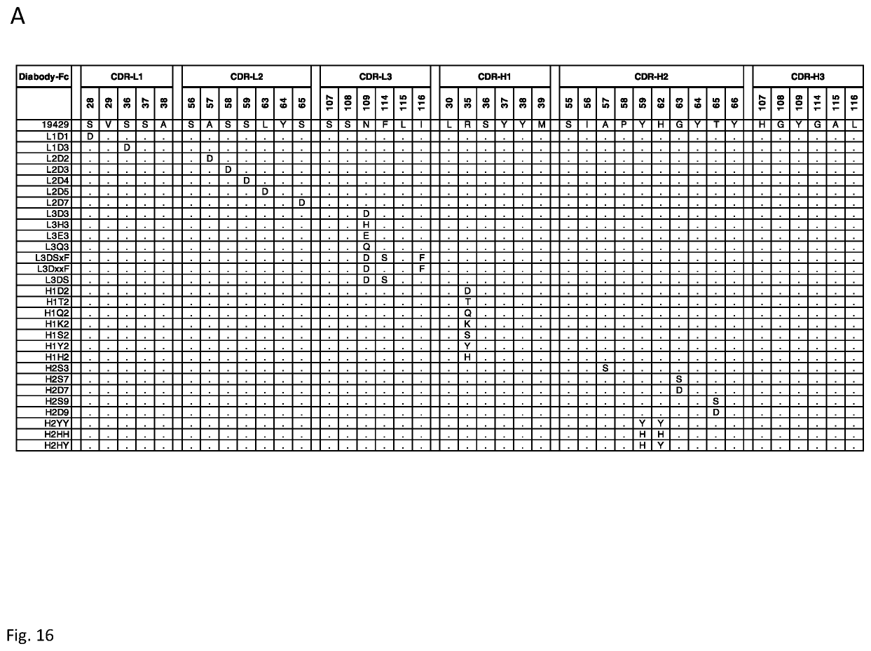

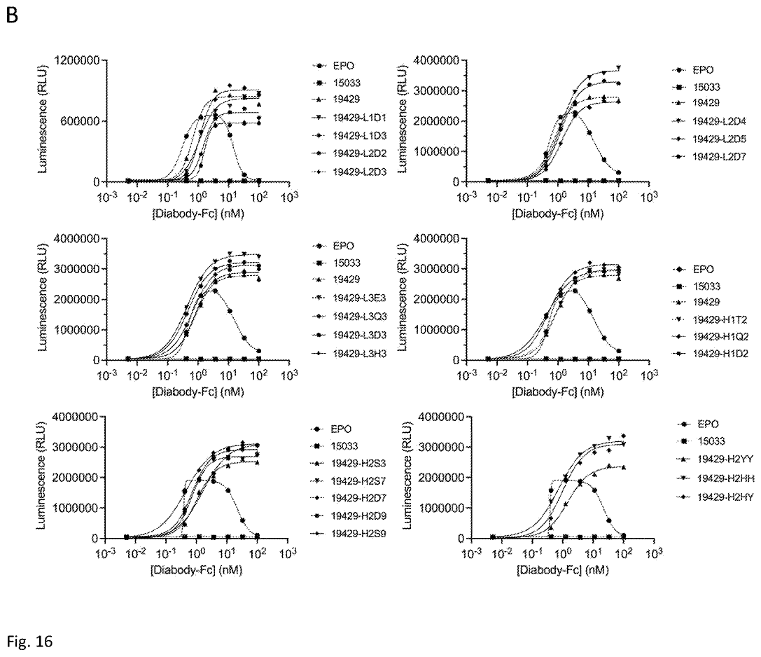

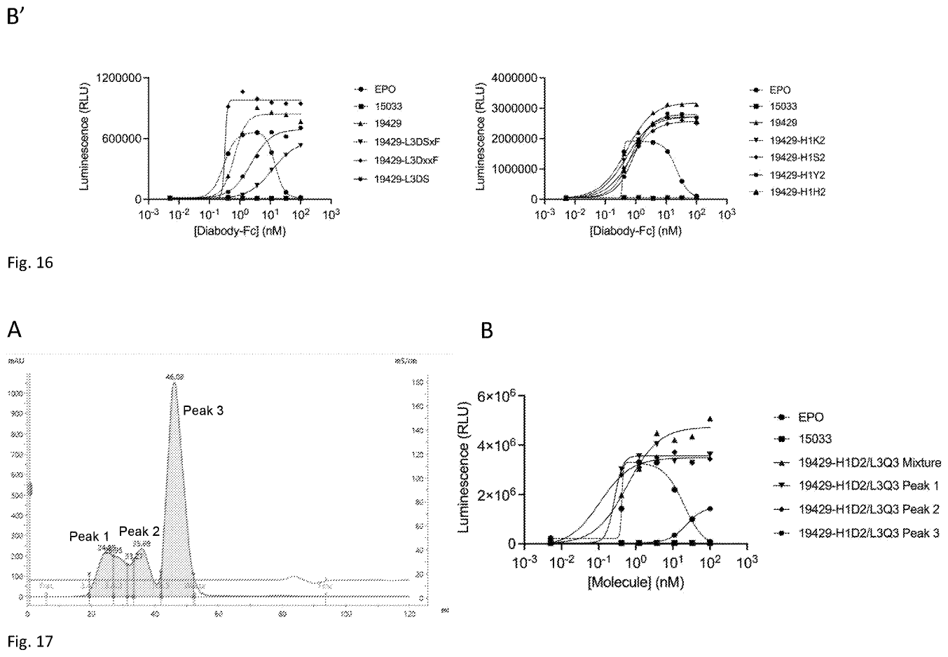

The affinity maturation randomization scheme left several positions unchanged that may have an impact on agonistic function. These positions are generally non-binding based on our structural analysis. For example, Arg35 in CDR-H1 of 14949 was never mutated (FIG. 13). These individual positions were mutated to hydrophilic residues and subsequently tested for activity in a UT7/EPO cell proliferation assay (FIGS. 16A and 16B). In general, hydrophilic substitutions at these positions were very well tolerated. The only exceptions were changes in CDR-L3 of 19429 where two variants—the L3DSxF and the L3DS—showed a significant reduction in hEPOR activation in this assay.

### Testing Activity of Higher-Order Species of 19113 and 19429

The 19429-H1D2/L3Q3 double variant was used to carry out a more detailed analysis on the activity profile of the various higher-order species that are present in the protein-A purified material (FIGS. 17A and 17B). Size exclusion chromatography (SEC) was used to separate this mixture, which consisted of 3 peaks that very likely corresponded to 3 different species of varying molecular weights (FIG. 17A). Using a UT7/EPO cell proliferation assay, Peak 3, which is the major species and most likely to be the diabody-Fc monomer, had the lowest activity (FIG. 17B). Peaks 1 and 2, which correspond to the molecular weights of the dimer and tetramer, respectively, possessed much higher activity. This data indicates that valency is a critical determinant of agonistic biological function.

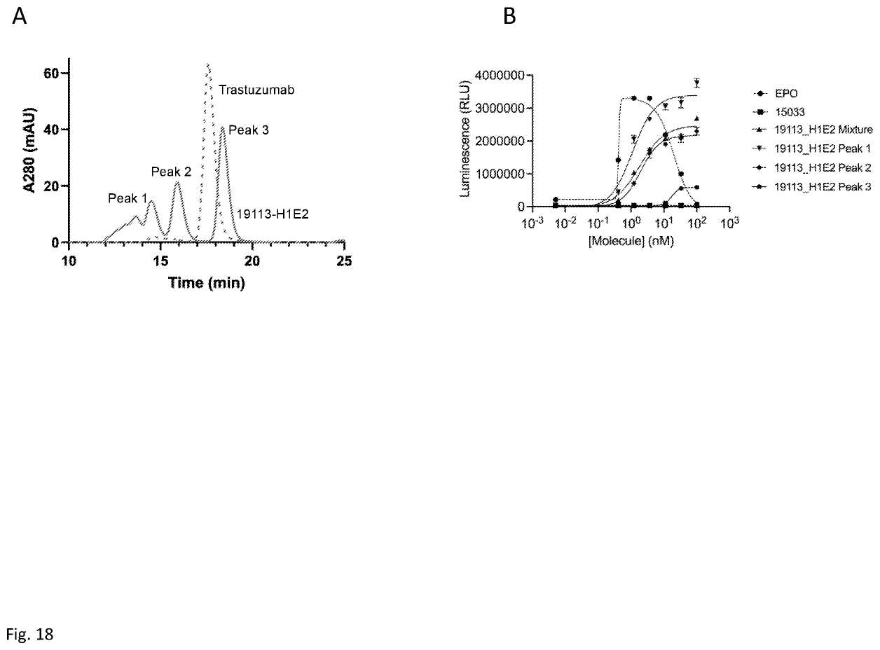

This was also tested using 19113-H1E2 variant. SEC identified 3 distinct species of varying molecular weights corresponding to a tetramer (Peak 1), dimer (Peak 2), and monomer (Peak 3) (FIG. 18A). Functional analysis using a UT7/EPO cell proliferation assay revealed that the monomeric species had the lowest activity (FIG. 18B). These results indicate that the engineering of agonists must consider the valency of the target engagement and will impact process development.

Taken together, the functional and structural characterization of the current invention underscore its novel mechanism of EPOR activation and stimulation of erythropoiesis. This mechanism involves inducing an asymmetric receptor geometry that while closely resembling that of the natural EPO ligand in its penultimate form, remains unique in that it involves a discontinuous, non-natural, and non-obvious receptor binding mechanism that is not found in biology or all previous art.

### REFERENCES

1. Persson, H., Ye, W., Wernimont, A., Adams, J. J., Koide, A., Koide, S., Lam, R. & Sidhu, S. S. CDR-H3 diversity is not required for antigen recognition by synthetic antibodies. J Mol Biol 425, 803-11 (2013).
2. Liu, Z., Stoll, V. S., Devries, P. J., Jakob, C. G., Xie, N., Simmer, R. L., Lacy, S. E., Egan, D. A., Harlan, J. E., Lesniewski, R. R. & Reilly, E. B. A potent erythropoietin-mimicking human antibody interacts through a novel binding site. Blood 110, 2408-13 (2007).
3. Goncalves, F., Lacout, C., Feger, F., Cohen-Solal, K., Guichard, J., Cramer, E., Vainchenker, W. & Dumenil, D. Inhibition of erythroid differentiation and induction of megakaryocytic differentiation by thrombopoietin are regulated by two different mechanisms in TPO-dependent UT-7/c-mpl and TF-1/c-mpl cell lines. Leukemia 12, 1355-66 (1998).
4. Livnah, O., Stura, E. A., Johnson, D. L., Middleton, S. A., Mulcahy, L. S., Wrighton, N.C., Dower, W. J., Jolliffe, L. K. & Wilson, I. A. Functional mimicry of a protein hormone by a peptide agonist: the EPO receptor complex at 2.8 A. Science 273, 464-71 (1996).
5. Moraga, I., Wernig, G., Wilmes, S., Gryshkova, V., Richter, C. P., Hong, W. J., Sinha, R., Guo, F., Fabionar, H., Wehrman, T. S., Krutzik, P., Demharter, S., Plo, I., Weissman, I. L., Minary, P., Majeti, R., Constantinescu, S. N., Piehler, J. & Garcia, K. C. Tuning Cytokine Receptor Signaling by Re-orienting Dimer Geometry with Surrogate Ligands. Cell 160, 1196-208 (2015).
6. Nelson, B. & Sidhu, S. S. Synthetic antibody libraries. Methods Mol Biol 899, 27-41 (2012).
7. Syed, R., Reid, S., Li, C. et al. Efficiency of signalling through cytokine receptors depends critically on receptor orientation. Nature 395, 511-516 (1998).
8. Lim, A. R., Ketchem, R. R., Borges, L. et al. Diversity of Antibody Epitopes Can Induce Signaling through the Erythropoietin Receptor. Biochemistry 49, 18, 3797-3804 (2010).
9. Yu, X., Orr, C. M., Chan, H. T. C. et al. Reducing affinity as a strategy to boost immunomodulatory antibody agonism. Nature 614, 539-547 (2023).

1. A selective activation region of the human erythropoietin receptor (hEPOR) that provides for selective activation of the erythropoiesis-specific effects of the hEPOR, the selective activation region comprises a first epitope, the first epitope comprising residues 82, 84 to 88 and 90 of the hEPOR, wherein numbers refer to SEQ ID NO: 29.

2. The selective activation region of claim 1 further comprising a second epitope, the second epitope comprising residues 119, 121, 123, 125, 128, 129, 131, 134 and 136 of hEPOR.

3. The selective activation region of claim 1 comprising residues 82, 84 to 88, 90, 119, 121, 123, 125, 128, 129, 131, 134, and 136 of hEPOR.

4. The selective activation region of claim 2 wherein the first epitope comprises QEDEPWL (SEQ ID NO: 1).

5. The selective activation region of claim 3 wherein the second epitope comprises PERTSGPHV (SEQ ID NO. 2).

6. An antibody or antigen-binding portion thereof that binds to a selective activation region of the human erythropoietin receptor (hEPOR) that provides for selective activation of the erythropoiesis-specific effects of the hEPOR, said activation region being according to claim 1.

7. An antibody or antigen-binding portion thereof that binds to human erythropoietin receptor (hEPOR) comprising a CDR-L1 region having a contiguous amino acid sequence X1X2X3X4X5, wherein:
X1 is S, D or T;
X2 is V or an aliphatic amino acid;
X3 and X4 are D, E, G, H, K, N, Q, R, or S; and
X5 is A or an aliphatic amino acid.

8. (canceled)

9. The antibody or antigen-binding portion of claim 7 further comprising a CDR-L2 having a contiguous amino acid sequence X1X2X3X4X5X6X7, wherein:
X1 is S or T;
X2 is A, D or aliphatic amino acid;
X3 and X4 are D, E, G, H, K, N, Q, R, or S;
X5 is L, D or aliphatic amino acid;
X6 is Y or polar amino acid; and
X7 is S, D or T.

10. (canceled)

11. The antibody or antigen-binding portion of claim 7 further comprising a CDR-L3 having a contiguous amino acid sequence X1X2X3X4X5X6, wherein:
X1 is S, F, T, A, I, or P;
X2 is S, P, T, C, A, F, Y, G, R or D,
X3 is Y, R, P, S, D, R, H, F, N, I, G, P, E, Q or T;
X4 is S, F, A, G, V, Y, P, T, or N;
X5 is L, P or aliphatic amino acid; and
X6 is I, F or hydrophobic amino acid.

12. (canceled)

13. The antibody or antigen-binding portion of claim 7 further a CDR-H1 having a contiguous amino acid sequence X1X2X3X4X5X6, wherein:
X1 is L, F or aliphatic amino acid;
X2 is Y, S, N, G D, H, R, F, T, D, P, Q, K or I;
X3 is S, A, F, Y, R, N, G, T, D, or H;
X4 is Y, F, H, S, F, or N;
X5 is Y, A, F, V, L, G, P, T, S or an aliphatic or aromatic amino acid; and
X6 is I, M or hydrophobic amino acid.

14. (canceled)

15. The antibody or antigen-binding portion of claim 7 further comprising a CDR-H2 having a contiguous amino acid sequence X1X2X3X4X5X6X7X8X9X10, wherein: X1 is S, Y or T;
X2 is I or aliphatic amino acid;
X3 is S, Y, A or polar amino acid;
X4 is P or aliphatic amino acid;
X5 is Y, H, F or polar amino acid;
X6 is Y, S, H or polar amino acid;
X7 is S, T, D or G;
X8 is Y, F or polar amino acid;
X9 is T, D or S amino acid; and
X10 is Y, S or polar amino acid.

16. (canceled)

17. The antibody or antigen-binding portion of claim 7 further comprising a CDR-H3 having a contiguous amino acid sequence X1X2X3X4X5X6, wherein:
X1 is H, R or N;
X2 is G, A, V or S;
X3 is Y, F, or H;
X4 is G, S, I, V, A, T or aliphatic amino acid; and
X5 is A, G or aliphatic amino acid; and
X6 is M, L or hydrophobic amino acid.

18. (canceled)

19. The antibody or antigen-binding portion of claim 7 wherein the CDR-L1 comprising the contiguous amino acid sequence SVSSA (SEQ ID NO: 3).

20. The antibody or antigen-binding portion of claim 9 wherein the CDR-L2 comprising the contiguous amino acid sequence SASSLYS (SEQ ID NO: 4).

21. The antibody or antigen-binding portion of claim 11 wherein the CDR-L3 comprising the contiguous amino acid sequence SSYSLI (SEQ ID NO: 5) or the contiguous amino acid sequence AYWPI (SEQ ID NO: 6), or the contiguous amino acid sequence SSYSLF (SEQ ID NO: 24), or the contiguous amino acid sequence SSDSLF (SEQ ID NO: 25), or the contiguous amino acid sequence SSNFLI (SEQ ID NO: 30), or the contiguous amino acid sequence SSQFLI (SEQ ID NO: 36).

22. The antibody or antigen-binding portion of claim 13 wherein the CDR-H1 comprising the contiguous amino acid sequence LYSYYI (SEQ ID NO: 7), or the contiguous amino acid sequence LRSYYM (SEQ ID NO: 38) or the contiguous amino acid sequence LSSYYI (SEQ ID NO: 8), or the contiguous amino acid sequence LESYYJ (SEQ ID NO: 37), or the contiguous amino acid sequence LDSYYI (SEQ ID NO: 35), or the contiguous amino acid sequence LESYYM (SEQ ID NO: 34).

23. The antibody or antigen-binding portion of claim 15 wherein the CDR-H2 comprises the contiguous amino acid sequence SISPYYSYTY (SEQ ID NO: 9), or the contiguous amino acid sequence SISPHYGYTY (SEQ ID NO: 26), or the contiguous amino acid sequence SIAPYHGYTY (SEQ ID NO: 31).

24. The antibody or antigen-binding portion of claim 17 wherein the CDR-H3 comprises the contiguous amino acid sequence HGYGAM (SEQ ID NO: 10) or the contiguous amino acid sequence HSYAAL (SEQ ID NO: 11), or the contiguous amino acid sequence HGFGAM (SEQ ID NO: 27), or the contiguous amino acid sequence HGYSAM (SEQ ID NO: 28), or the contiguous amino acid sequence HGYGAL (SEQ ID NO: 32).

25. The antibody or antigen-binding portion of claim 6, wherein
a) the CDR-L1 comprises SEQ ID NO: 3, the CDR-L2 comprises SEQ ID NO: 4, the CDR-L3 comprises SEQ ID NO: 30, the CDR-H1 comprises SEQ ID NO: 38, the CDR-H2 comprises SEQ ID NO: 31, the CDR-H3 comprises SEQ ID NO: 32,
b) the CDR-L1 comprises SEQ ID NO: 3, the CDR-L2 comprises SEQ ID NO: 4, the CDR-L3 comprises SEQ ID NO: 25, the CDR-H1 comprises SEQ ID NO: 38, the CDR-H2 comprises SEQ ID NO: 26, the CDR-H3 comprises SEQ ID NO: 28,
c) the CDR-L1 comprises SEQ ID NO: 3, the CDR-L2 comprises SEQ ID NO: 4, the CDR-L3 comprises SEQ ID NO: 36, the CDR-H1 comprises SEQ ID NO: 38, the CDR-H2 comprises SEQ ID NO: 31, the CDR-H3 comprises SEQ ID NO: 32;
d) the CDR-L1 comprises SEQ ID NO: 3, the CDR-L2 comprises SEQ ID NO: 4, the CDR-L3 comprises SEQ ID NO: 5 the CDR-H1 comprises SEQ ID NO: 7, the CDR-H2 comprises SEQ ID NO: 9, the CDR-H3 comprises SEQ ID NO: 10;
e) the CDR-L1 comprises SEQ ID NO: 3, the CDR-L2 comprises SEQ ID NO: 4, the CDR-L3 comprises SEQ ID NO: 6, the CDR-H1 comprises SEQ ID NO: 8, the CDR-H2 comprises SEQ ID NO: 9, the CDR-H3 comprises SEQ ID NO: 11;
f) the CDR-L1 comprises SEQ ID NO: 3, the CDR-L2 comprises SEQ ID NO: 4, the CDR-L3 comprises SEQ ID NO: 5, the CDR-H1 comprises SEQ ID NO: 38, the CDR-H2 comprises SEQ ID NO: 9, the CDR-H3 comprises SEQ ID NO: 10; or
g) the CDR-L1 comprises SEQ ID NO: 3, the CDR-L2 comprises SEQ ID NO: 4, the CDR-L3 comprises SEQ ID NO: 25, the CDR-H1 comprises SEQ ID NO: 37, the CDR-H2 comprises SEQ ID NO: 26, the CDR-H3 comprises SEQ ID NO: 28.

26. (canceled)

27. (canceled)

28. (canceled)

29. (canceled)

30. (canceled)

31. (canceled)

32. (canceled)

33. The antibody or antigen-binding portion thereof of claim 6, wherein the antibody or antigen-binding portion is a diabody, a Fab, a F(ab)2, a tetravalents, or a multivalent.

34. A method of treating kidney disease or anemia in a subject comprising administering to the subject the antibody or antigen-binding portion thereof of claim 6.


SEQUENCE LISTING (38 sequences)

SEQ ID NO: 1 - protein, 7 aa, Homo sapiens
QEDEPWL

SEQ ID NO: 2 - protein, 9 aa, Homo sapiens
PERTSGPHV

SEQ ID NO: 3 - protein, 5 aa, synthetic construct
SVSSA

SEQ ID NO: 4 - protein, 7 aa, synthetic construct
SASSLYS

SEQ ID NO: 5 - protein, 6 aa, synthetic construct
SSYSLI

SEQ ID NO: 6 - protein, 5 aa, synthetic construct
AYWPI

SEQ ID NO: 7 - protein, 6 aa, synthetic construct
LYSYYI

SEQ ID NO: 8 - protein, 6 aa, synthetic construct
LSSYYI

SEQ ID NO: 9 - protein, 10 aa, synthetic construct
SISPYYSYTY

SEQ ID NO: 10 - protein, 6 aa, synthetic construct
HGYGAM

SEQ ID NO: 11 - protein, 6 aa, synthetic construct
HSYAAL

SEQ ID NO: 12 - protein, 5 aa, synthetic construct
GGGGG

SEQ ID NO: 13 - protein, 9 aa, synthetic construct
IKGGGGGEV

SEQ ID NO: 14 - protein, 9 aa, synthetic construct
LKVLSRGVV

SEQ ID NO: 15 - protein, 9 aa, synthetic construct
ISARAGSLV

SEQ ID NO: 16 - protein, 9 aa, synthetic construct
SKSRAGGEV

SEQ ID NO: 17 - protein, 9 aa, synthetic construct
LKGGRGGKV

SEQ ID NO: 18 - protein, 9 aa, synthetic construct
NKGGGGAKV

SEQ ID NO: 19 - protein, 9 aa, synthetic construct
IKGSSRDDI

SEQ ID NO: 20 - protein, 9 aa, synthetic construct
MKHAGRGGV

SEQ ID NO: 21 - protein, 9 aa, synthetic construct
SKGGGGGEV

SEQ ID NO: 22 - protein, 9 aa, synthetic construct
MKHAGRGGV

SEQ ID NO: 23 - protein, 9 aa, synthetic construct
LECSDCSGI

SEQ ID NO: 24 - protein, 6 aa, synthetic construct
SSNFLI

SEQ ID NO: 25 - protein, 6 aa, synthetic construct
SSDSLF

SEQ ID NO: 26 - protein, 10 aa, synthetic construct
SISPHYGYTY

SEQ ID NO: 27 - protein, 6 aa, synthetic construct
HGFGAM

SEQ ID NO: 28 - protein, 6 aa, synthetic construct
HGYSAM

SEQ ID NO: 29 - protein, 508 aa, Homo sp.
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 30 - protein, 6 aa, synthetic construct
SSNFLI

SEQ ID NO: 31 - protein, 10 aa, synthetic construct
SIAPYHGYTY

SEQ ID NO: 32 - protein, 6 aa, synthetic construct
HGYGAL

SEQ ID NO: 33 - skipped

SEQ ID NO: 34 - protein, 6 aa, synthetic construct
LESYYM

SEQ ID NO: 35 - protein, 6 aa, synthetic construct
LDSYYM

SEQ ID NO: 36 - protein, 6 aa, synthetic construct
SSQFLI

SEQ ID NO: 37 - protein, 6 aa, synthetic construct
LESYYI

SEQ ID NO: 38 - protein, 6 aa, synthetic construct
LRSYYM